(19) United States
(12) Patent Application Publication
(10) Pub. No.: US 2016/0256333 A1
(43) Pub. Date: Sep. 8, 2016

(54) Method for Manufacturing Absorbent Articles Including a Discrete Barrier Member

(71) Applicant: The Procter & Gamble Company, Cincinnati, OH (US)

(72) Inventors: Darrell Ian Brown, Mason, OH (US); Ronald Herbert Helton, Cincinnati, OH (US)

(21) Appl. No.: 15/059,375

(22) Filed: Mar. 3, 2016

Related U.S. Application Data
(60) Provisional application No. 62/129,050, filed on Mar. 6, 2015.

(51) Int. Cl. A61F 13/15

(52) U.S. Cl. CPC A61F 13/15804; A61F 13/15764; A61F 13/15723; A61F 13/15747

(57) ABSTRACT

A method for manufacturing an absorbent article including a topsheet, a backsheet, a core, a discrete barrier member, a first cuff, and a second cuff. A continuous barrier substrate may be transferred onto a folding roll. The folding roll may form a fold in at least a portion of the continuous barrier substrate. The continuous barrier substrate may then be cut into a discrete barrier member including a leading edge portion, an opposing trailing edge portion, and a central portion therebetween. The discrete barrier member may be disposed on at least a portion of a first cuff substrate and a second cuff substrate. A topsheet substrate may associate with at least a portion of the discrete barrier member, the first cuff substrate, and the second cuff substrate.

### CROSS REFERENCE TO RELATED APPLICATIONS

This application claims the benefit of U.S. Provisional Application No. 62/129,050 filed on Mar. 6, 2015, which is herein incorporated herein by reference.

### FIELD OF THE INVENTION

The present disclosure relates to methods for manufacturing absorbent articles, and more particularly, methods for manufacturing absorbent articles including a discrete barrier member.

### BACKGROUND OF THE INVENTION

Along an assembly line, diapers and various types of other absorbent articles may be assembled by adding components to and otherwise modifying an advancing, continuous web of material. For example, in some processes, advancing webs of material are combined with other advancing webs of material. In other examples, individual components created from advancing webs of material are combined with advancing webs of material, which in turn, are then combined with other advancing webs of material. Webs of material and component parts used to manufacture diapers may include: backsheets, topsheets, absorbent cores, front and/or back ears, fastener components, and various types of webs and components such as leg elastics, barrier leg cuff elastics, and waist elastics. Once the desired component parts are assembled, the advancing web(s) and component parts are subjected to a final knife cut to separate the web(s) into discrete diapers or other absorbent articles. The discrete diapers or absorbent articles may also then be folded and packaged.

As mentioned above, during the assembly process, component parts such as elastics and other materials are used to manufacture diapers. Generally, a number of component parts have been added to absorbent articles to improve the fit of the absorbent article, to reduce leakage of the absorbent article, and to reduce irritation to the skin of the wearer of the absorbent article. To accomplish these objectives, absorbent articles having an opening that provides a passageway to void space for collected feces and urine have been proposed. Similarly, absorbent articles having a member to create separate areas for urine and feces have also been proposed.

However, it has been found that these absorbent articles are expensive to manufacture due to the added number of component parts. For example, elastics and additional non-woven materials have been added to the absorbent article to create a barricade for feces and urine. Further, these absorbent articles are difficult to manufacture due to the complexity of adding additional component parts to partition the absorbent article while maintaining relatively high manufacturing speeds.

In addition, some absorbent articles that currently provide a means for separating feces and urine are inadequate. For example, it has been found that current separating means may fail to provide the desired tension across the absorbent article to separate bodily exudates. More specifically, once the absorbent article has been placed on the wearer, the separating means have been found to slump or bunch such that the member loses contact with the wearer and provides an opening to allow feces and urine to move uninhibited throughout the absorbent article.

Thus, a need exists for improved methods of manufacturing absorbent articles including discrete barrier members that separate feces and urine and are more easily maintained in close contact with the wearer's body.

### SUMMARY OF THE INVENTION

Aspects of the present disclosure relate to a method for assembling absorbent articles. The method may include the following steps: advancing a first cuff substrate and a second cuff substrate in a machine direction, wherein the first cuff substrate comprises a first inner cuff edge and a first outer cuff edge and the second cuff substrate comprises a second inner cuff edge and a second outer cuff edge, and wherein the first inner cuff edge is separated from the second inner cuff edge in a cross direction by a first distance; advancing a continuous barrier substrate in the machine direction to an infeed device; advancing the infeed device and the continuous barrier substrate at a first velocity; rotating a folding roll at a second velocity about an axis of rotation, wherein the second velocity is greater than the first velocity; advancing the continuous barrier substrate onto the folding roll, wherein the folding roll comprises one or more grooves; rotating the folding member about an axis of rotation; cutting the continuous barrier substrate to form a discrete barrier member, wherein the barrier member comprises a leading edge portion, a trailing edge portion opposite the leading edge portion, and a central portion between the leading edge portion and the trailing edge portion; forcing a portion of the discrete barrier member into the one or more grooves of the folding roll; connecting the trailing edge portion of the discrete barrier member with the first cuff substrate and the second cuff substrate; extending the trailing edge portion of the discrete barrier member in the cross direction by separating the first cuff substrate and the second cuff substrate such that the first inner cuff edge is separated from the second inner cuff edge in the cross direction by a second distance, wherein the second distance is greater than the first distance; advancing a topsheet substrate having a first topsheet edge and a second topsheet edge opposite from the first topsheet edge in the cross direction; and connecting the leading edge portion of the discrete barrier member with the topsheet substrate.

In another embodiment, a method for manufacturing an absorbent article, wherein the absorbent article comprises a topsheet, a backsheet, a core, a discrete barrier member, a first cuff, and a second cuff may include the following steps: advancing a first cuff substrate and a second cuff substrate in a machine direction, wherein the first cuff substrate comprises a first inner cuff edge and an opposing first outer cuff edge, and wherein the second cuff substrate comprises a second inner cuff edge and an opposing second outer cuff edge, and wherein each of the first cuff substrate and the second cuff substrate comprise a first cuff surface and a second cuff surface such that each include an inner edge region, an opposing outer edge region, and a central region therebetween; folding the first cuff substrate such that the first inner cuff edge is associated with at least one of the first edge region and the central region of the first cuff surface to form a first cuff fold, wherein the first cuff fold comprises a first fold edge; folding the second cuff substrate such that the second inner cuff edge is associated with at least one of the first edge region and the central region of the first cuff surface to form a second cuff fold, wherein the second cuff fold comprises a second fold edge, wherein the first fold edge is separated by the second fold edge by a first distance in a cross direction; advancing a continuous barrier substrate in the machine direction onto an infeed device, wherein the infeed device advances the continuous barrier substrate at a first velocity; advancing the continuous barrier substrate onto an outer circumferential surface of a folding roll, wherein the folding roll is configured to rotate about an axis of rotation at a second velocity, and wherein the second velocity is greater than the first velocity; cutting the continuous barrier substrate on the folding roll to form a discrete barrier member, wherein the discrete barrier member comprises a leading edge portion, a trailing edge portion opposite the leading edge portion, and a central portion between the leading edge portion and the trailing edge portion; folding a portion of the discrete barrier substrate; connecting the trailing edge portion of the discrete barrier member with the first cuff substrate and the second cuff substrate; advancing a topsheet substrate having a first topsheet edge opposite from the second topsheet edge in the cross direction, wherein the first topsheet edge and the second topsheet edge extend longitudinally in the machine direction; and connecting the leading edge portion of the discrete barrier member with the topsheet substrate.

In yet another embodiment, a method for manufacturing an absorbent article, wherein the absorbent article comprises a topsheet, a backsheet, a core, a discrete barrier member, a first cuff, and a second cuff may include the following steps: advancing a continuous barrier substrate in a machine direction; positioning the continuous barrier substrate on a transfer member of an infeed device, wherein the transfer member is configured to advancing the continuous barrier substrate at a first velocity; advancing a portion of the continuous barrier substrate onto an outer circumferential surface of a folding roll, wherein the portion of the continuous barrier substrate slips along the outer circumferential surface of the folding roll; rotating the folding roll about an axis of rotation at a second velocity, wherein the second velocity is greater than the first velocity; cutting the continuous barrier substrate to form a discrete barrier member; advancing the discrete barrier substrate about the axis of rotation of the folding roll and folding a portion of the discrete barrier substrate; advancing a cuff substrate onto a bond roll; disposing the discrete barrier substrate on at least a portion of the cuff substrate; connecting a trailing edge portion of the discrete barrier member with the cuff substrate; associating a topsheet substrate with at least a portion of the discrete barrier member and the cuff substrate; and connecting a leading edge portion of the discrete barrier member with the topsheet substrate.

### BRIEF DESCRIPTION OF THE DRAWINGS

FIG. 1 is a perspective view of a diaper pant;

FIG. 2 is a partially cut away plan view of the diaper pant shown in FIG. 1;

FIG. 3A is a cross-sectional view of the diaper pant of FIG. 2 taken along line 3A-3A in accordance with one non-limiting embodiment of the present disclosure;

FIG. 3B is a cross-sectional view of the diaper pant of FIG. 2 taken along line 3B-3B in accordance with one non-limiting embodiment of the present disclosure;

FIG. 4 is a partially cut away plan view of a diaper in accordance with one non-limiting embodiment of the present disclosure;

FIG. 5A is a schematic representation of a process used to manufacture absorbent articles comprising a discrete barrier member in accordance with one non-limiting embodiment of the present disclosure;

FIG. 5B is a schematic representation of a portion of the process used to manufacture absorbent articles comprising a discrete barrier member in accordance with one non-limiting embodiment of the present disclosure;

FIG. 5C is a schematic representation of a portion of the process used to manufacture absorbent articles comprising a discrete barrier member in accordance with one non-limiting embodiment of the present disclosure;

FIG. 6A is a perspective view of a first cuff substrate and a second cuff substrate of FIG. 5A taken along line 6A-6A in accordance with one non-limiting embodiment of the present disclosure;

FIG. 6B is a perspective view of a first cuff substrate and a second cuff substrate of FIG. 5A taken along line 6B-6B in accordance with one non-limiting embodiment of the present disclosure;

FIG. 6C is a perspective view of a first cuff substrate and a second cuff substrate of FIG. 5B taken along line 6C-6C in accordance with one non-limiting embodiment of the present disclosure;

FIG. 6D is a perspective view of a first cuff substrate and a second cuff substrate of FIG. 5B taken along line 6C-6C in accordance with one non-limiting embodiment of the present disclosure;

FIG. 6E is a perspective view of a first cuff substrate and a second cuff substrate of FIG. 5B taken along line 6E-6E in accordance with one non-limiting embodiment of the present disclosure;

FIG. 7 is a perspective view of a discrete barrier member in accordance with one non-limiting embodiment of the present disclosure;

FIG. 8A is a schematic representation of a process used to manufacture absorbent articles comprising a discrete barrier member in accordance with one non-limiting embodiment of the present disclosure;

FIG. 8B is a schematic representation of a process used to manufacture absorbent articles comprising a discrete barrier member in accordance with one non-limiting embodiment of the present disclosure;

FIG. 9 is a schematic representation of a process used to manufacture absorbent articles comprising a discrete barrier member in accordance with one non-limiting embodiment of the present disclosure;

FIG. 10 is a perspective view of a discrete barrier member disposed on a first cuff substrate and a second cuff substrate in accordance with one non-limiting embodiment of the present disclosure;

FIG. 11A is a schematic representation of a process used to manufacture absorbent articles comprising a discrete barrier member in accordance with one non-limiting embodiment of the present disclosure;

FIG. 11B is a schematic representation of a process used to manufacture absorbent articles comprising a discrete barrier member in accordance with one non-limiting embodiment of the present disclosure;

FIG. 12 is a perspective view of a topsheet substrate disposed on a discrete barrier member, and a portion of the first cuff substrate and the second cuff substrate in accordance with one non-limiting embodiment of the present disclosure;

FIG. 13A is a perspective view of a topsheet substrate in accordance with one non-limiting embodiment of the present disclosure;

FIG. 13B is a perspective view of a topsheet substrate having a compressed topsheet width in accordance with one non-limiting embodiment of the present disclosure;

FIG. 13C is a perspective view of a topsheet substrate having a compressed topsheet width in accordance with one non-limiting embodiment of the present disclosure;

FIG. 14 is a perspective view of a topsheet substrate disposed on a discrete barrier member, and a portion of the first cuff substrate and the second cuff substrate in accordance with one non-limiting embodiment of the present disclosure;

FIG. 15 is a top view of a topsheet substrate disposed on a discrete barrier member, and a portion of the first cuff substrate and the second cuff substrate in accordance with one non-limiting embodiment of the present disclosure; and

FIG. 16 is a perspective view of a topsheet substrate disposed on a discrete barrier member, and a portion of the first cuff substrate and the second cuff substrate in accordance with one non-limiting embodiment of the present disclosure.

### DETAILED DESCRIPTION OF THE INVENTION

The following term explanations may be useful in understanding the present disclosure:

“Absorbent article” is used herein to refer to consumer products whose primary function is to absorb and retain soils and wastes. “Diaper” is used herein to refer to an absorbent article generally worn by infants and incontinent persons about the lower torso. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant may be configured with a continuous or closed waist opening and at least one continuous, closed, leg opening prior to the article being applied to the wearer.

“Longitudinal” means a direction running substantially perpendicular from a waist edge to a longitudinally opposing waist edge of an absorbent article when the article is in a flat out, uncontracted state, or from a waist edge to the bottom of the crotch, i.e. the fold line, in a bi-folded article. Directions within 45 degrees of the longitudinal direction are considered to be “longitudinal.” “Lateral” refers to a direction running from a longitudinally extending side edge to a laterally opposing longitudinally extending side edge of an article and generally at a right angle to the longitudinal direction. Directions within 45 degrees of the lateral direction are considered to be “lateral.”

“Substrate” is used herein to describe a material which is primarily two-dimensional (i.e. in an XY plane) and whose thickness (in a Z direction) is relatively small (i.e. 1/10 or less) in comparison to the substrate's length (in an X direction) and width (in a Y direction). Non-limiting examples of substrates include a web, layer or layers or fibrous materials, nonwovens, films and foils such as polymeric films or metallic foils. These materials may be used alone or may comprise two or more layers joined together. As such, a web is a substrate.

“Nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

The term “machine direction” (MD) is used herein to refer to the direction of material flow through a process. In addition, relative placement and movement of material may be described as flowing in the machine direction through a process from upstream in the process to downstream in the process.

The term “cross direction” (CD) is used to herein refer to the direction perpendicular to the direction of material flow through a process. The cross direction may be substantially perpendicular to the machine direction.

The present disclosure relates to methods for manufacturing absorbent articles including a discrete barrier member. More particularly, the methods are directed to manufacturing an absorbent article including a topsheet, a backsheet, a core, a discrete barrier member, a first cuff and a second cuff. As discussed in more detail below, the methods may include attaching the discrete barrier member to the first cuff and the second cuff. The attachment of the discrete barrier member to the first and second cuffs may allow the barrier member to provide the desired tension across the absorbent article to maintain contact with the wearer during use and to maintain adequate separation of feces and urine in the absorbent article. Further to the above, the discrete barrier member may be attached to the topsheet of the absorbent article. The attachment of the barrier member to the topsheet may allow the discrete barrier member to remain in contact with the topsheet and, thus, to maintain the separation of the feces and urine once disposed on the absorbent article.

As discussed in more detail below, the methods according to the present disclosure may be utilized in the production of various components of absorbent articles, such as diapers. To help provide additional context to the subsequent discussion of the process embodiments, the following provides a general description of absorbent articles in the form of diapers that include components including the materials that may be used by the methods and apparatuses discussed herein.

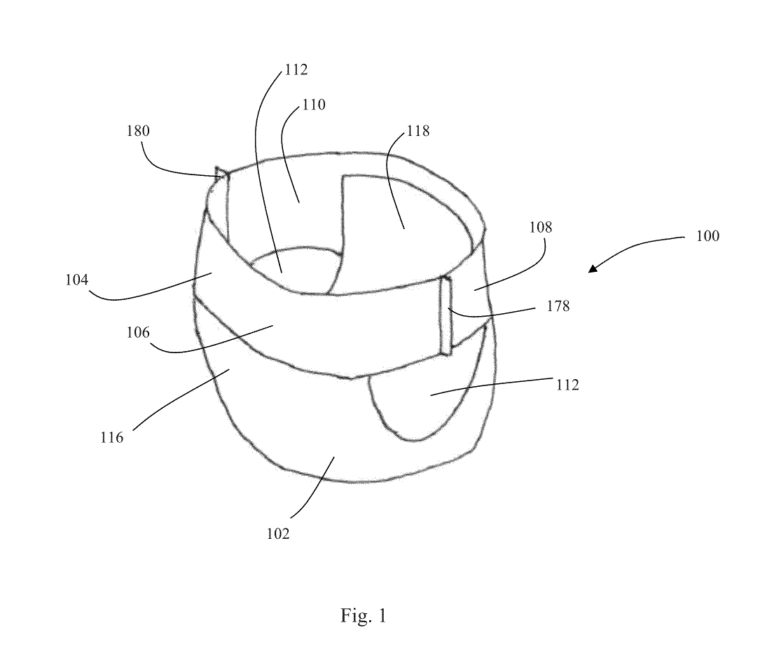

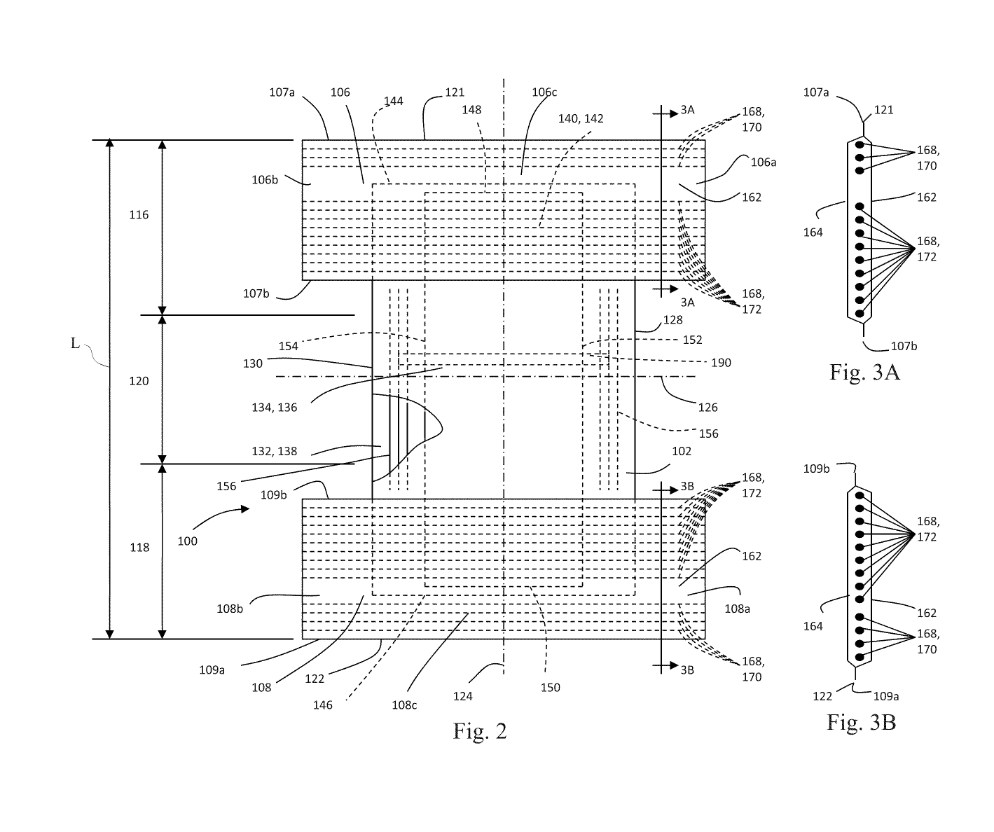

FIGS. 1, 2, and 4 illustrate an example of an absorbent article 100, such as a diaper, that may be assembled with the methods discussed herein. In particular, FIG. 1 shows a perspective view of an absorbent article 100 in a pre-fastened configuration, and FIG. 2 shows a plan view of the absorbent article 100 with the portion of the diaper that faces away from a wearer oriented towards the viewer. The absorbent article 100 shown in FIGS. 1 and 2 includes a chassis 102 and a ring-like elastic belt 104. As discussed below in more detail, a first elastic belt 106 and a second elastic belt 108 are connected together to form the ring-like elastic belt 104.

With continued reference to FIG. 2, the chassis 102 includes a first waist region 116, a second waist region 118, and a crotch region 120 disposed intermediate the first and second waist regions. The first waist region 116 may be configured as a front waist region, and the second waist region 118 may be configured as back waist region. In some embodiments, the length of each of the front waist region, back waist region, and crotch region 120 may be ⅓ of the length of the absorbent article 100. The diaper 100 may also include a laterally extending front waist edge 121 in the front waist region 116 and a longitudinally opposing and laterally extending back waist edge 122 in the back waist region 118. To provide a frame of reference for the present discussion, the absorbent article 100 and chassis 102 of FIG. 2 is shown with a longitudinal axis 124 and a lateral axis 126. In some embodiments, the longitudinal axis 124 may extend through the front waist edge 121 and through the back waist edge 122. And the lateral axis 126 may extend through a first longitudinal or right side edge 128 and through a midpoint of a second longitudinal or left side edge 130 of the chassis 102.

As shown in FIGS. 1, 2, and 4, the absorbent article 100 may include an inner, body facing surface 132, and an outer, garment facing surface 134. The chassis 102 may include a backsheet 136 and a topsheet 138. The chassis 102 may also include an absorbent assembly 140 including an absorbent core 142 that may be disposed between a portion of the topsheet 138 and the backsheet 136. As discussed in more detail below, the absorbent article 100 may also include other features, such as leg elastics and/or leg cuffs to enhance the fit around the legs of the wearer.

As shown in FIG. 2, the periphery of the chassis 102 may be defined by the first longitudinal side edge 128, a second longitudinal side edge 130; a first laterally extending end edge 144 disposed in the first waist region 116; and a second laterally extending end edge 146 disposed in the second waist region 118. Both side edges 128 and 130 extend longitudinally between the first end edge 144 and the second end edge 146. When the absorbent article 100 is worn on the lower torso of a wearer, the front waist edge 121 and the back waist edge 122 of the chassis 102 may encircle a portion of the waist of the wearer. At the same time, the chassis side edges 128 and 130 may encircle at least a portion of the legs of the wearer. Moreover, the crotch region 120 may be generally positioned between the legs of the wearer with the absorbent core 142 extending from the front waist region 116 through the crotch region 120 to the back waist region 118.

It is also to be appreciated that a portion or the whole of the absorbent article 100 may also be made laterally extensible. The additional extensibility may help allow the absorbent article 100 to conform to the body of a wearer during movement by the wearer. The additional extensibility may also help, for example, allow the diaper 100, including a chassis 102 having a particular size before extension, to extend in the front waist region 116, the back waist region 118, or both waist regions of the diaper 100 and/or chassis 102 to provide additional body coverage for wearers of differing size, i.e., to tailor the diaper to an individual wearer. Such extension of the waist region or regions may give the absorbent article a generally hourglass shape, so long as the crotch region is extended to a relatively lesser degree than the waist region or regions, and may impart a tailored appearance to the article when it is worn.

As previously mentioned, the diaper 100 may include a backsheet 136. The backsheet 136 may also define the outer surface 134 of the chassis 102. The backsheet 136 may be impervious to fluids (e.g., menses, urine, and/or runny feces) and may be manufactured from a thin plastic film, although other flexible liquid impervious materials may also be used. The backsheet 136 may prevent the exudates absorbed and contained in the absorbent core from wetting articles which contact the diaper 100, such as bedsheets, pajamas, and undergarments. The backsheet 136 may also include a woven or nonwoven material, polymeric films such as thermoplastic films of polyethylene or polypropylene, and/or a multi-layer or composite materials comprising a film and a nonwoven material (e.g., having an inner film layer and an outer nonwoven layer). The backsheet may also include an elastomeric film. An example backsheet 136 may be a polyethylene film having a thickness of from about 0.012 mm (0.5 mils) to about 0.051 mm (2.0 mils). Exemplary polyethylene films are manufactured by Clopay Corporation of Cincinnati, Ohio, under the designation BR-120 and BR-121 and by Tredegar Film Products of Terre Haute, Ind., under the designation XP-39385. The backsheet 136 may also be embossed and/or matte-finished to provide a more clothlike appearance. Further, the backsheet 136 may permit vapors to escape from the absorbent core (i.e., the backsheet is breathable) while still preventing exudates from passing through the backsheet 136. The size of the backsheet 136 may be dictated by the size of the absorbent core 142 and/or particular configuration or size of the diaper 100.

Also described above, the absorbent article 100 may include a topsheet 138. The topsheet 138 may also define all or part of the inner surface 132 of the chassis 102. The topsheet 138 may be compliant, soft feeling, and non-irritating to the wearer's skin. It may be elastically stretchable in one or two directions. Further, the topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art.

Topsheets 138 may be selected from high loft nonwoven topsheets, apertured film topsheets, and apertured nonwoven topsheets. Apertured film topsheets may be pervious to bodily exudates, yet substantially non-absorbent, and have a reduced tendency to allow fluids to pass back through and rewet the wearer's skin. Exemplary apertured films may include those described in U.S. Pat. Nos. 5,628,097; 5,916,661; 6,545,197; and 6,107,539.

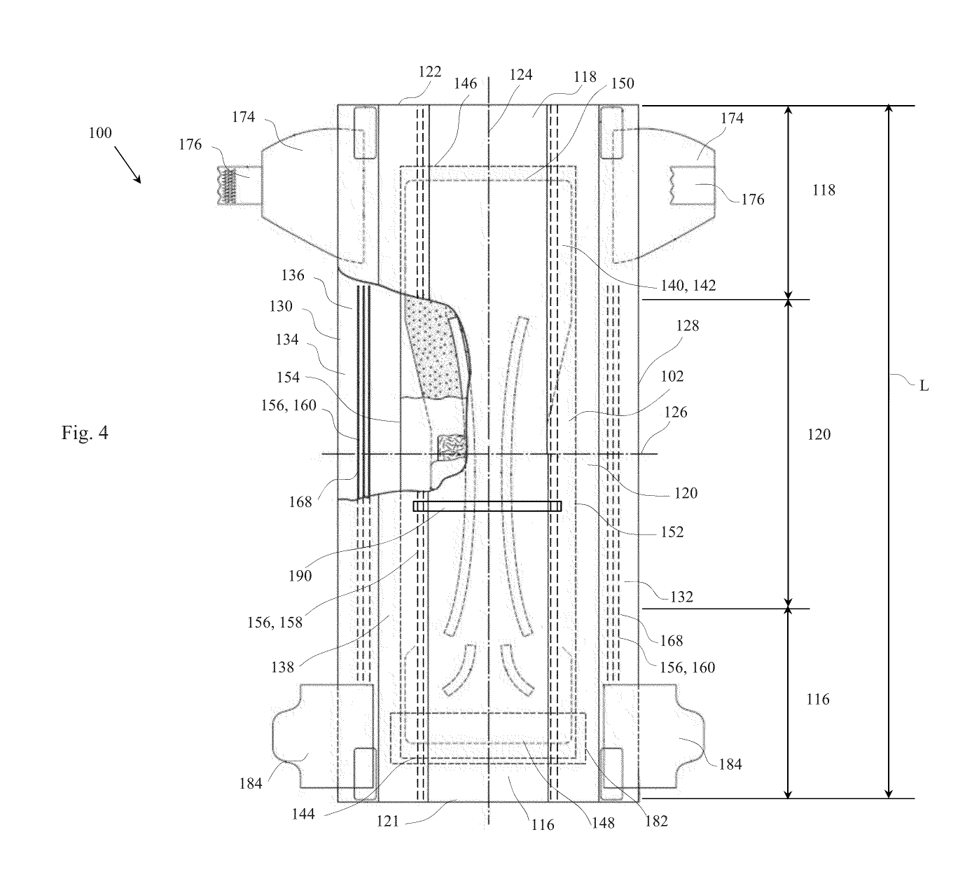

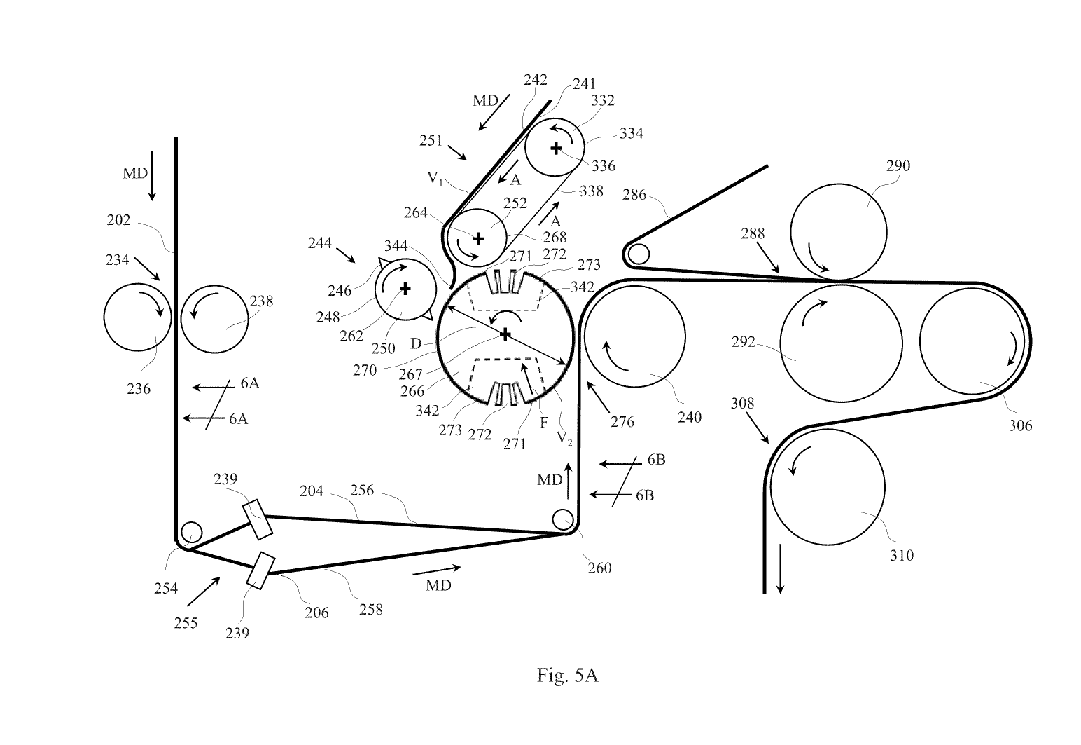

The absorbent article 100 may also include an absorbent assembly 140 that is joined to the chassis 102. As shown in FIGS. 2 and 4, the absorbent assembly 140 may have a laterally extending front edge 148 in the front waist region 116 and may have a longitudinally opposing and laterally extending back edge 150 in the back waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the diaper. Exemplary absorbent structures for use as the absorbent core of the present disclosure are described in U.S. Pat. Nos. 4,610,678; 4,673,402; 4,888,231; and 4,834,735.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprise primarily absorbent gelling material in amounts of at least about 60%, 70%, 80%, 85%, 90%, 95%, or even about 100%, where the remainder of the core may comprise a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 and 2004/0097895.

The absorbent article 100 may also include elasticized leg cuffs 156. It is to be appreciated that the leg cuffs 156 may be and are sometimes also referred to as leg bands, side flaps, barrier cuffs, elastic cuffs, or gasketing cuffs. The elasticized leg cuffs 156 may be configured in various ways to help reduce the leakage of body exudates in the leg regions. For example, in some embodiments, a gasketing leg cuff 160 may be positioned adjacent to the side edge 130, 128 of the chassis 102 and a barrier leg cuff 158 may be positioned between a gasketing leg cuff 160 and the longitudinal axis 124 of the absorbent article 100. Example leg cuffs 156 may include those described in U.S. Pat. Nos. 3,860,003; 4,909,803; 4,695,278; 4,795,454; 4,704,115; 4,909,803; U.S. Patent Publication No. 2009/0312730 A1; and U.S. Patent Publication No. 2013/0255865 A1.

As mentioned above, diaper pants may be manufactured with a ring-like elastic belt 104 and provided to consumers in a configuration wherein the front waist region 116 and the back waist region 118 are connected to each other as packaged, prior to being applied to the wearer.

As such, the absorbent article may have a continuous perimeter waist opening 110 and continuous perimeter leg openings 112 such as shown in FIG. 1. As previously mentioned, the ring-like elastic belt 104 is defined by a first elastic belt 106 connected with a second elastic belt 108. As shown in FIG. 2, the first elastic belt 106 defines first and second opposing end regions 106a, 106b and a central region 106c, and the second elastic 108 belt defines first and second opposing end regions 108a, 108b and a central region 108c.

The central region 106c of the first elastic belt is connected with the first waist region 116 of the chassis 102, and the central region 108c of the second elastic belt 108 is connected with the second waist region 118 of the chassis 102. As shown in FIG. 1, the first end region 106a of the first elastic belt 106 is connected with the first end region 108a of the second elastic belt 108 at first side seam 178, and the second end region 106b of the first elastic belt 106 is connected with the second end region 108b of the second elastic belt 108 at second side seam 180 to define the ring-like elastic belt 104 as well as the waist opening 110 and leg openings 112.

As shown in FIGS. 2, 3A, and 3B, the first elastic belt 106 also defines an outer lateral edge 107a and an inner lateral edge 107b, and the second elastic belt 108 defines an outer lateral edge 109a and an inner lateral edge 109b. The outer lateral edges 107a, 109a may also define the front waist edge 121 and the laterally extending back waist edge 122. The first elastic belt and the second elastic belt may also each include an outer, garment facing layer 162 and an inner, wearer facing layer 164. It is to be appreciated that the first elastic belt 106 and the second elastic belt 108 may comprise the same materials and/or may have the same structure. In some embodiments, the first elastic belt 106 and the second elastic belt may comprise different materials and/or may have different structures. It should also be appreciated that the first elastic belt 106 and the second elastic belt 108 may be constructed from various materials. For example, the first and second belts may be manufactured from materials such as plastic films; apertured plastic films; woven or nonwoven webs of natural materials (e.g., wood or cotton fibers), synthetic fibers (e.g., polyolefins, polyamides, polyester, polyethylene, or polypropylene fibers) or a combination of natural and/or synthetic fibers; or coated woven or nonwoven webs. In some embodiments, the first and second elastic belts may include a nonwoven web of synthetic fibers, and may include a stretchable nonwoven. In other embodiments, the first and second elastic belts may include an inner hydrophobic, non-stretchable nonwoven material and an outer hydrophobic, non-stretchable nonwoven material.

The first and second elastic belts 106, 108 may also each include belt elastic material interposed between the outer layer 162 and the inner layer 164. The belt elastic material may include one or more elastic elements such as strands, ribbons, or panels extending along the lengths of the elastic belts. As shown in FIGS. 2, 3A, and 3B, the belt elastic material may include a plurality of elastic strands 168 that may be referred to herein as outer, waist elastics 170 and inner, waist elastics 172.

As shown in FIG. 2, the outer, waist elastics 170 extend continuously laterally between the first and second opposing end regions 106a, 106b and across the central region 106c of the first elastic belt 106 and between the first and second opposing end regions 108a, 108b and across the central region 108c of the second elastic belt 108. In some embodiments, some elastic strands 168 may be configured with discontinuities in areas. For example, as shown in FIG. 2, the inner, waist elastics 172 extend intermittently along the first and second elastic belts 106, 108. More particularly, the inner, waist elastics 172 extend along the first and second opposing end regions 106a, 106b and partially across the central region 106c of the first elastic belt 106. The inner, waist elastics 172 also extend along the first and second opposing end regions 108a, 108b and partially across the central region 108c of the second elastic belt 108. As such, the inner, waist elastics 172 do not extend across the entirety of the central regions 106c, 108c of the first and second elastic belts 106, 108. Thus, some elastic strands 168 may not extend continuously through regions of the first and second elastic belts 106, 108 where the first and second elastic belts 106, 108 overlap the absorbent assembly 140. In some embodiments, some elastic strands 168 may partially extend into regions of the first and second elastic belts 106, 108 where the first and second elastic belts 106, 108 overlap the absorbent assembly 140. In some embodiments, some elastic strands 168 may not extend into any region of the first and second elastic belts 106, 108 where the first and second elastic belts 106, 108 overlap the absorbent assembly 140. It is to be appreciated that the first and/or second elastic belts 106, 108 may be configured with various configurations of discontinuities in the outer, waist elastics 170 and/or the inner, waist elastic elastics 172.

In some embodiments, the elastic strands 168 may be disposed at a constant interval in the longitudinal direction. In other embodiments, the elastic strands 168 may be disposed at different intervals in the longitudinal direction. As discussed in more detail below, the belt elastic strands 168, in a stretched condition, may be interposed and joined between the uncontracted outer layer and the uncontracted inner layer. When the belt elastic material is relaxed, the belt elastic material returns to an unstretched condition and contracts the outer layer and the inner layer. The belt elastic material may provide a desired variation of contraction force in the area of the ring-like elastic belt. It is to be appreciated that the chassis 102 and elastic belts 106, 108 may be configured in different ways other than as depicted in FIG. 2.

Referring to FIG. 4, in some embodiments, the absorbent article 100 may include a fastening system. The fastening system can be used to provide lateral tensions about the circumference of the absorbent article to hold the absorbent article on the wearer. The fastening system may comprise a fastener such as tape tabs, hook and loop fastening components, interlocking fasteners such as tabs and slots, buckles, buttons, snaps, and/or hermaphroditic fastening components. A landing zone 182 may be provided on the front waist region 116 for at least a portion of the fastener to be releasably attached to. Exemplary fastening systems may include those described in U.S. Pat. Nos. 3,848,594; 4,662,875; 4,846,815; 4,894,060; 4,946,527; 5,151,092; and 5,221,274.

As illustrated in FIG. 4, the absorbent article 100 may comprise front ears 184 and back ears 174. The front ears 184 and the back ears 174 may be an integral part of the chassis 102. For example, the front ears 184 and the back ears 174 may be formed from the topsheet 138 and/or the backsheet 136. Alternatively, the front ears 184 and the back ears 174 may be attached to the backsheet 136 and/or the topsheet 138. The front ears 184 and the back ears 174 may be extensible to facilitate attachment on the landing zone 182 and to maintain placement around the waist of the wearer. The back ears 174 may comprise a tab member 176. The tab member 176 may be attached to a portion of the back ears 174 to facilitate attachment to the landing zone 182.

The absorbent article 100 may also comprise a discrete barrier member 190, as illustrated in FIGS. 2 and 4. An exemplary discrete barrier member may include that described in U.S. Patent Application No. 61/918,954; 61/919,067; 61/918,966; and 61/918,978. The discrete barrier member 190 may be positioned in at least one of the crotch region 120, the front waist region 116, and the back waist region 118 of the absorbent article 100. More specifically, the discrete barrier member 190 may be positioned a distance from the front edge 121 of the absorbent article 100. The distance may be 20% to 50% and/or 30% to 45% of the total length L of the absorbent article 100 taken from the front edge 121 to the rear edge 122 of the absorbent article 100. The discrete barrier member 190 may be positioned such that it extends substantially perpendicular to the longitudinal axis 124 of the absorbent article 100. Similarly, the discrete barrier member 190 may be positioned such that it extends substantially parallel to the lateral axis 126 of the absorbent article 100. The discrete barrier member 190 may be extensible in at least one of the longitudinal direction and the lateral direction.

As previously mentioned, the methods according to the present disclosure may be utilized to assemble discrete absorbent articles 100 and/or various components of absorbent articles 100, such as for example, chassis 102, elastic belts 106, 108, leg cuffs 156, and/or discrete barrier members 190. Although the following methods may be provided in the context of absorbent articles 100, as shown in FIGS. 1, 2, and 4, it is to be appreciated that the methods and apparatuses herein may be used with various process configurations and/or absorbent articles, such as for example, disclosed in U.S. Pat. No. 7,569,039; U.S. Patent Publication Nos. 2005/0107764 A1, 2012/0061016 A1, and 2012/0061015 A1; 2013/0255861 A1; 2013/0255862 A1; 2013/0255863 A1; 2013/0255864 A1; and 2013/0255865 A1.

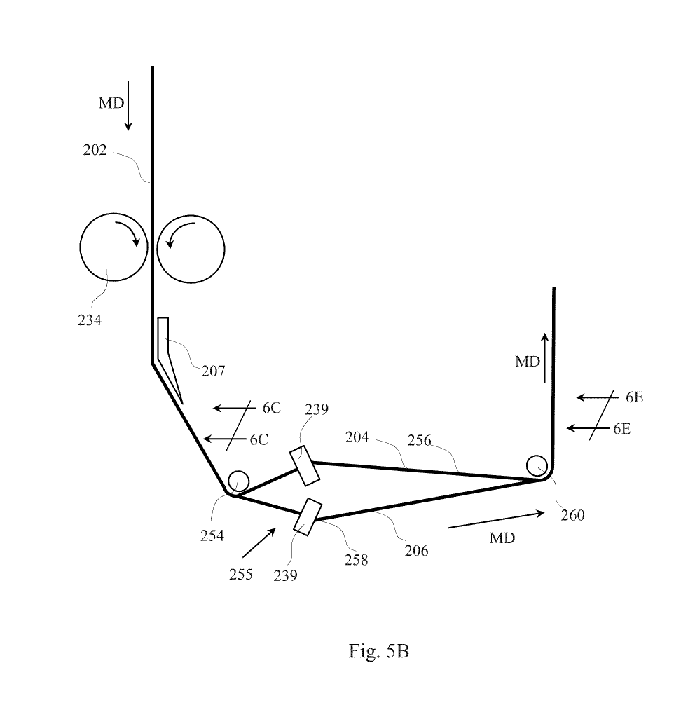

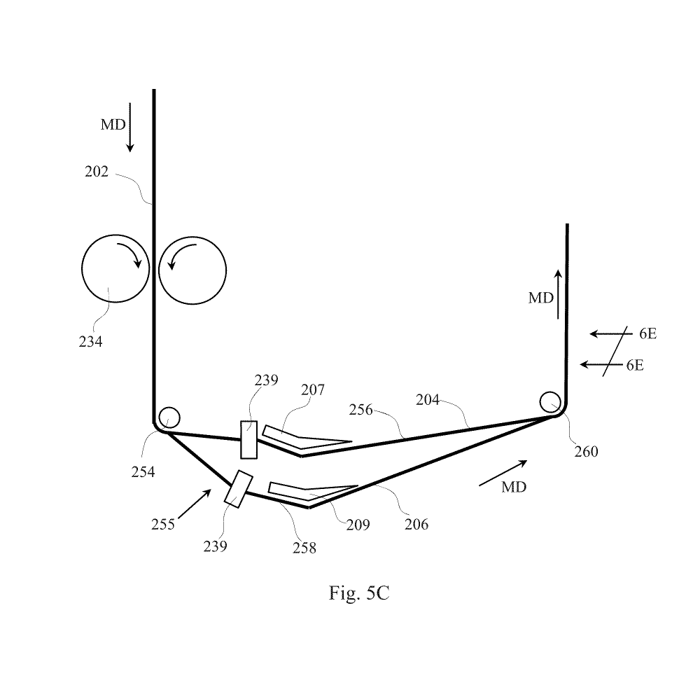

FIG. 5A shows an exemplary schematic representation of a method that may be used to manufacture an absorbent article 100, such as previously described, including a discrete barrier member 190. Generally, the method may include advancing a cuff substrate 202 in a machine direction MD. The cuff substrate 202 may be used to form leg cuffs 156, for example, as shown in FIGS. 2 and 4. The cuff substrate 202 may be slit into a first cuff substrate 204 and a second cuff substrate 206. The first cuff substrate 204 and the second cuff substrate 206 may be advanced to a repositioning device 255. The repositioning device 255 may separate the first cuff substrate 204 from the second cuff substrate 206 at a desired distance in the cross direction, referred to herein as a first distance. Once the first cuff substrate 204 and the second cuff substrate 206 have been repositioned, the first and second cuff substrates 204, 206 may be advanced to a first bonding area 276.

In addition, a continuous barrier substrate 242 may be advanced in the machine direction MD to a folding roll 266. The continuous barrier substrate 242 may be disposed on the folding roll 266. As the folding roll 266 rotates, the continuous barrier substrate 242 may be advance to a cutting device 244. The cutting device 244 may cut the continuous barrier substrate 244 to form a discrete barrier member 190. The discrete barrier member 190 may be advanced to the first bonding area 276. At the first bonding area 276, the discrete barrier member 190 may be disposed on the first cuff substrate 204 and the second cuff substrate 206. In the first bonding area 276, a portion of the discrete barrier member 190 may be connected to at least a portion of the first cuff substrate 204 and the second cuff substrate 206. Upon exiting the first bonding area 276, the discrete barrier member 190, first cuff substrate 204, and second cuff substrate 206 may be advanced to accept a topsheet substrate 286. The topsheet substrate 286 may be disposed on at least a portion of the discrete barrier member 190, the first cuff substrate 204, and the second cuff substrate 206. Once the topsheet substrate 286 has been disposed on the discrete barrier member 190 and the first and second cuff substrates 204, 206, these substrates may be advanced through a second bond area 288 and a third bond area 308. The second and third boding areas 288, 308, may bond any one of the topsheet substrate 286, the first cuff substrate 204, the second cuff substrate 206, and the discrete barrier member 190. This process will be described in more detail herein.

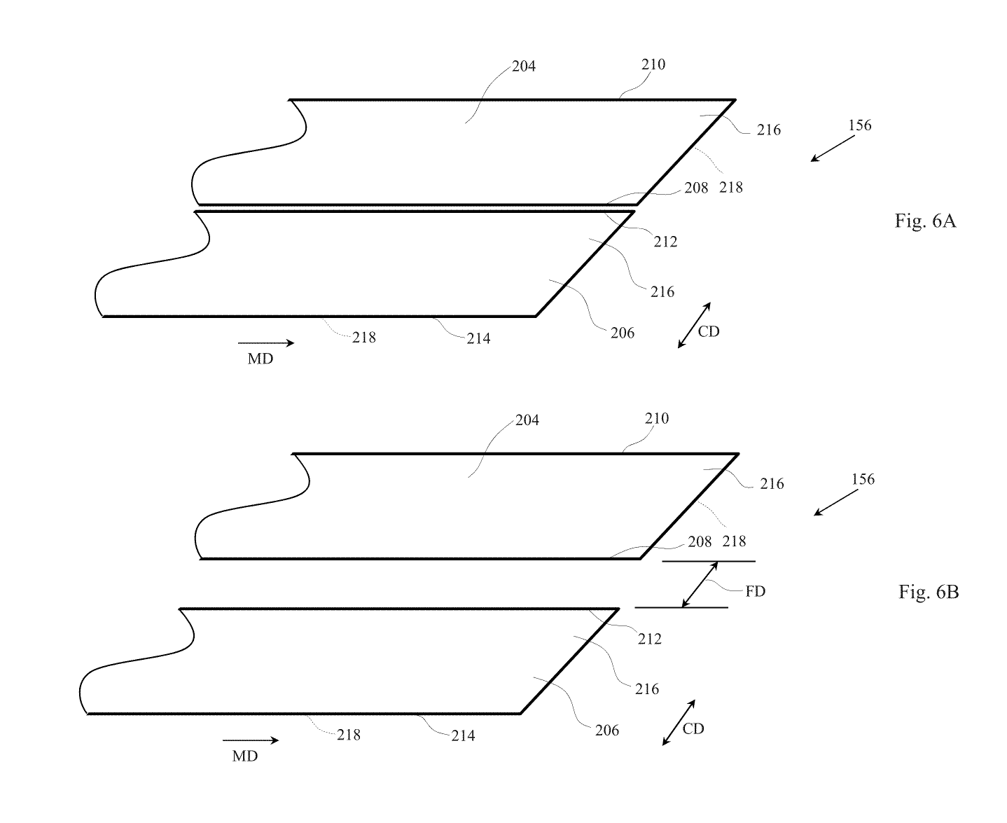

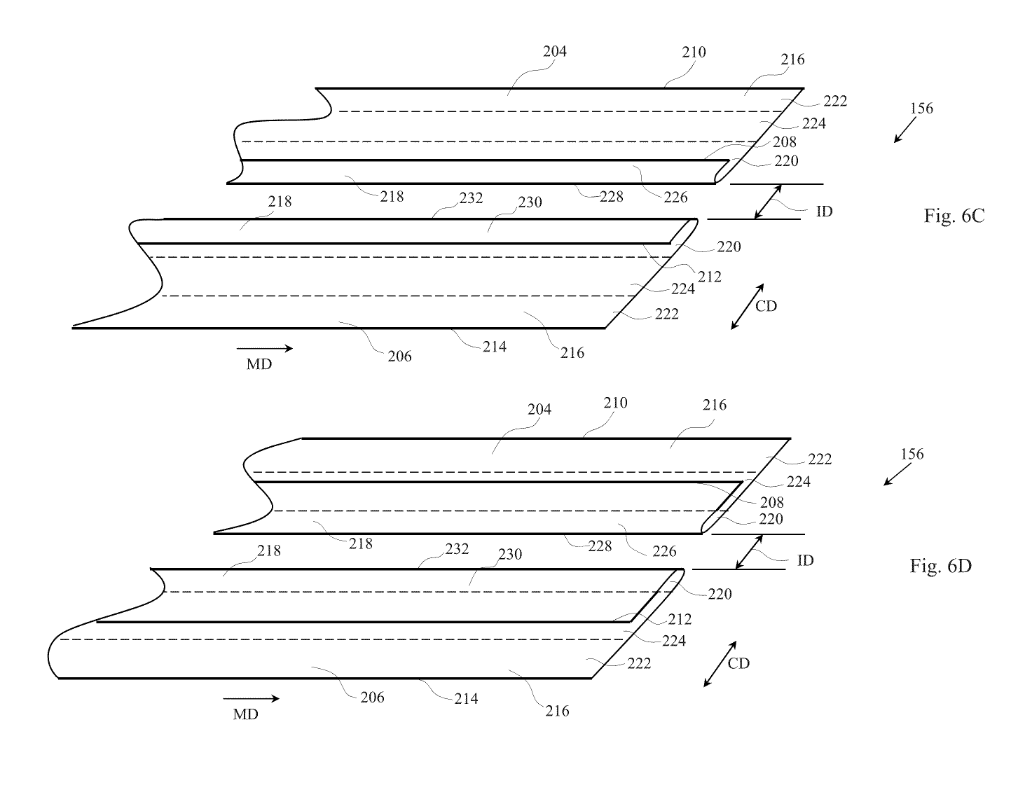

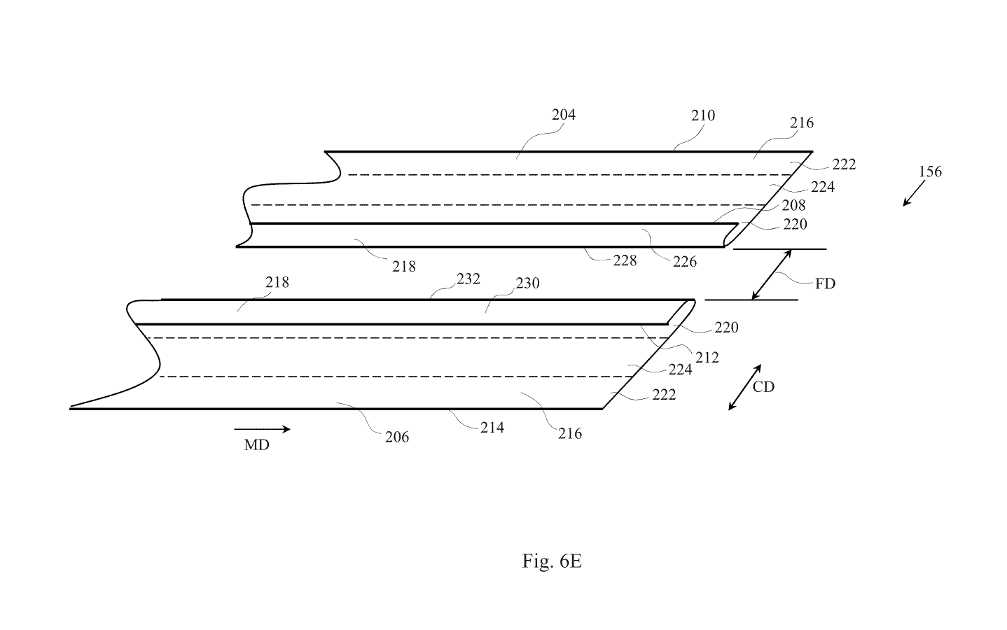

As previously described, the cuff substrate 202 may be separated by a slitting device 234, as shown in FIG. 5A. The slitting device 234 may include a slitting roll 236 including one or more blades extending radially outward and an anvil roll 238 including an anvil. Other slitting devices may be used such as those available from Tidland Products, Camas, Wash. Any deceive which separates the cuff substrate 202 may be used as the slitting device 234. The slitting device 234 may separate the cuff substrate 202 into a first cuff substrate 204 and a second cuff substrate 206, as illustrated in FIG. 6A.

Still referring to FIG. 6A, the first cuff substrate 204 may include a first inner cuff edge 208 and a first outer cuff edge 210, opposite the first inner cuff edge 208. The second cuff substrate 206 may include a second inner cuff edge 212 and a second outer cuff edge 214, opposite the second inner cuff edge 212. The first and second inner cuff edges 208, 212 and the first and second outer cuff edges 210, 214 extend in a direction substantially parallel to the machine direction MD. Further, the first cuff substrate 204 and the second cuff substrate 206 may include a first cuff surface 216 and a second cuff surface 218. It is to be appreciated that one or more elastics may be disposed on each cuff substrate to form a leg cuff 156, as previously discussed. Further, it is also to be appreciated that an additional substrate may be disposed on the one or more elastics. However, FIG. 6A is a simplified schematic representation of the first and second cuff substrates.

It is to be appreciated that the first inner cuff edge 208 may not be the desired distance from the second inner cuff edge 212 after the cuff substrate 202 is slit. Stated another way, the first cuff substrate 204 and the second cuff substrate may not be appropriately spaced for additional materials to be added to the first and second cuff substrates in subsequent processes. Thus, to adjust the spacing between the first inner cuff edge 208 and the second inner cuff edge 212, each of the first cuff substrate 204 and the second cuff substrate 206 may be directed to a separating device 255. The separating device 255, as shown in FIG. 5A, may reposition the first cuff substrate 204 and the second cuff substrate 206. More specifically, the first cuff substrate 204 and the second cuff substrate 206 may be advanced toward and, subsequently, diverge at a first roll 254. Each of the first cuff substrate 204 and the second cuff substrate 206 may pass through a canted idler 239, which allows the cuff substrates to diverge in the cross direction CD. Upon diverging, the first cuff substrate 204 may advance down a first lane 256 and the second cuff substrate 206 may advance down a second lane 258. Subsequently, the first cuff substrate 204 and the second cuff substrate 206 may be directed to converge at a second roll 260. The first cuff substrate 204 and the second cuff substrate 206 may be placed about the second roll 260 such that the first inner cuff edge 208 and the second inner cuff edge 212 may be separated by a first distance FD, as illustrated in FIG. 6B. The first distance FD may be from about 5 mm to about 100 mm and/or about 10 mm to about 85 mm and/or about 15 mm to about 65 mm and/or about 20 mm to about 45 mm, including all 0.5 mm increments therebetween. Once the first cuff substrate 204 and the second cuff substrate are placed in the desired position, the first cuff substrate 204 and the second cuff substrate 206 may be directed to a first bonding area 276.

In some embodiments, after slitting the cuff substrate 202, the first cuff substrate 204 and the second cuff substrate 206 may be advanced in the machine direction MD through a folding device 207, as illustrated in FIG. 5B. The folding device 207 may be adapted to fold the first cuff substrate 204 and the second cuff substrate 206, as illustrated, for example, in FIGS. 6C and 6D. In some exemplary embodiments, the first cuff substrate 204 and the second cuff substrate 206 may be folded to substantially surround one or more elastics disposed on the cuff substrate (not shown). The first cuff substrate 204 and the second cuff substrate 206 may include a first cuff surface 216 and a second cuff surface 218. The first cuff surface 216 and the second cuff surface 218 may each include an inner edge region 220, an outer edge region 222 opposite the inner edge region 220, and a central region 224 between the inner edge region 220 and the outer edge region 222. The first cuff substrate 204 may be folded such that the first inner cuff edge 208 is associated with the inner edge region 220 of the first cuff substrate 204, as shown in FIG. 6C. The folded first cuff substrate 204 may form a first cuff fold 226 that includes a first fold edge 228. Likewise, the second cuff substrate 206 may be folded such that the second inner cuff edge 212 is associated with the inner edge region 220 of the second cuff substrate 206, as shown in FIG. 6C. The folded second cuff substrate 206 may form a second cuff fold 230 that includes a second fold edge 232. The first cuff substrate 204 and the second cuff substrate 206 may be positioned such that the first fold edge 228 is an initial distance ID away from the second fold edge 232. In some embodiments, the initial distance ID may be from about 5 mm to about 45 mm and/or about 10 mm to about 35 mm and/or about 15 mm to about 25 mm, including all 0.5 mm increments therebetween. It is to be appreciated that the first cuff substrate 204 and the second cuff substrate may be folded in various ways and may include additional components, such as for example, in accordance with the methods and apparatuses disclosed in U.S. Patent Publication Nos. 2013/0255861 A1; 2013/0255862 A1; 2013/0255863 A1; 2013/0255864 A1; and 2013/0255865 A1.

FIG. 6D illustrates an exemplary embodiment of a folded first cuff substrate 204 and a second cuff substrate 206. The first cuff substrate 204 may be folded such that the first inner cuff edge 208 is associated with at least a portion of the central region 224 of the first cuff substrate 204. The folded first cuff substrate 204 may form a first cuff fold 226 that includes a first fold edge 228. Likewise, the second cuff substrate 206 may be folded such that the second inner cuff edge 212 is associated with at least a portion of the central region 224 of the second cuff substrate 206, as shown in FIG. 6D. The folded second cuff substrate 206 may form a second cuff fold 230 that includes a second fold edge 232. The first cuff substrate 204 and the second cuff substrate 206 may be positioned such that the first fold edge 228 is an initial distance away from the second fold edge 232. The initial distance ID may be from about 5 mm to about 45 mm and/or about 10 mm to about 35 mm and/or about 15 mm to about 25 mm, including all 0.5 mm increments therebetween.

It is to be appreciated that first cuff substrate 204 and the second cuff substrate 206 may be folded such that the first inner cuff edge 208 is associated with any one of the outer edge region 222, the central region 224, and the inner edge region. Further, the first cuff substrate 204 and the second cuff substrate may be folded such that each of the first inner cuff edge 208 and the second inner cuff 212 edge do not associate with the same region of the cuff surface. For example, the first cuff substrate 204 may be folded such that the first inner cuff edge 208 is associated with the outer edge region 222, and the second cuff substrate 204 may be folded such that the second inner cuff edge 212 is associated with the central region 224.

Referring to FIGS. 5B and 6E, the first fold edge 228 may not be the desired distance from the second fold edge 230 after each of the first cuff substrate 206 and the second cuff substrate 208 are separated and/or folded. Stated another way, the initial distance ID may not be the appropriate spacing between the first cuff substrate 204 and the second cuff substrate 206 for additional materials to be added to the first and second cuff substrates in subsequent processes. Thus, to adjust the spacing between the first fold edge 228 and the second fold edge 230, each of the first cuff substrate 204 and the second cuff substrate 206 may be directed to a separating device 255. The separating device 255 may include a first roll 254, a canted idler 239, a first lane 256, a second lane 258, and a second roll 260. More specifically, the first cuff substrate 204 and the second cuff substrate 206 may be advanced toward and, subsequently, diverge at a first roll 254 and pass over the canted idler 239. Upon diverging, the first cuff substrate 204 may advance down a first lane 256 and the second cuff substrate 206 may advance down a second lane 258. Subsequently, the first cuff substrate 204 and the second cuff substrate 206 may be directed to converge at a second roll 260. The first cuff substrate 204 and the second cuff substrate 206 may be placed about the second roll 260 such that the first fold edge 228 may be a first distance FD from the second fold edge 230, as illustrated in FIG. 6E. The first distance FD may be from about 5 mm to about 100 mm and/or about 10 mm to about 85 mm and/or about 15 mm to about 65 mm and/or about 20 mm to about 40 mm, including all 0.5 mm increments therebetween. Once the first cuff substrate 204 and the second cuff substrate are placed in the desired position, the first cuff substrate 204 and the second cuff substrate 206 may be directed to a first bonding area 276.

Referring to FIGS. 5C and 6E, as previously described, the initial distance ID may not be the appropriate spacing between the first cuff substrate 204 and the second cuff substrate 206 for additional materials to be added to the first and second cuff substrates in subsequent processes. Thus, to adjust the spacing between the first fold edge 228 and the second fold edge 230, each of the first cuff substrate 204 and the second cuff substrate 206 may be directed to a separating device 255. The separating device 255 may include a first roll 254, a canted idler 239, a first lane 256, a second lane 258, and a second roll 260. More specifically, the first cuff substrate 204 and the second cuff substrate 206 may be advanced toward and, subsequently, diverge at a first roll 254 and pass over the canted idler 239. Upon diverging, the first cuff substrate 204 may advance down a first lane 256 and the second cuff substrate 206 may advance down a second lane 258. Subsequently, each of the first cuff substrate 204 and the second cuff substrate 206 may be directed toward a folding device. The folding device may be a single device (not shown) that folds both of the first and second cuff substrate 204, 206 or multiple devices, as shown in FIG. 5C. As illustrated in FIG. 5C, the first cuff substrate 204 may be folded by a first folding device 207 and the second cuff substrate 206 may be folded by the second folding device 209. After being folded, the first cuff substrate 204 and the second cuff substrate 206 may be directed to converge at a second roll 260. The first cuff substrate 204 and the second cuff substrate 206 may be placed about the second roll 260 such that the first fold edge 228 may be a first distance FD from the second fold edge 230, as illustrated in FIG. 6E. The first distance FD may be from about 5 mm to about 100 mm and/or about 10 mm to about 85 mm and/or about 15 mm to about 65 mm and/or about 20 mm to about 40 mm, including all 0.5 mm increments therebetween. Once the first cuff substrate 204 and the second cuff substrate are placed in the desired position, the first cuff substrate 204 and the second cuff substrate 206 may be directed to a first bonding area 276.

It is to be appreciated that the first cuff substrate 204 and the second cuff substrate 206 may be positioned such that the first fold edge 228 is a desired distance from the second fold edge 232 after being slit and/or folded, and thus, repositioning the first cuff substrate 204 and the second cuff substrate 206 may be unnecessary. For example, the initial distance ID separating the first cuff substrate 204 and the second cuff substrate 206 may be equal to the first distance FD, or the desired distance for subsequent processes, and thus, need not undergo repositioning. In this instance, the first cuff substrate 204 and the second cuff substrate 206 may advance in the machine direction MD from at least one of the slitting device 236 and the folding device 207 to a first bonding area 276.

It is also to be appreciated that the first cuff substrate 204 and the second cuff substrate 206 need not be folded. The first cuff substrate 204 and the second cuff substrate 206 may proceed through the process unfolded, as shown in FIGS. 6A and 6B. However, the first cuff fold 226 and the second cuff fold 226 may provide additional strength for bonding, which will be described in more detail below. However, to be concise, the following illustrations and description will include that first cuff substrate 204 and the second cuff substrate 206 are folded.

Referring to FIG. 5A, in some embodiments, a continuous barrier substrate 242 may be advanced by an infeed device 251. The infeed device 251 may be used to regulate the advancement of the continuous barrier substrate 242. In some embodiments, the infeed device 251 may include, for example, a vacuum conveyor, a rotating vacuum drum, and/or a pair of compression nip rolls. It is to be appreciated that a continuous substrate may be fed in various ways and may include additional components, such as in accordance with the methods and apparatuses disclosed in U.S. Pat. Nos. 5,373,761; 5,693,165; 6,349,867; 6,596,108; 8,377,249; and U.S. Patent Publication Nos. 2014/0005021; 2014/0000798; 2014/0000795; and 2014/0000794. An example of an infeed device 251 for achieving control of an advancing continuous substrate 242 may be a cam device.

In some embodiments, for example, the infeed device 251 may include a first guide roll 252 including an outer circumferential surface 268 and configured to rotate about a central axis of rotation 264 and a second guide roll 332 including a second outer circumferential surface 334 and configured to rotate about a second central axis of rotation 336. The first guide roll 252 and the second guide roll 332 may be operatively connected by a transfer member 338, such as a belt. The transfer member 338 may surround a portion of the first guide roll 252 and the second guide roll 332 such that as the first guide roll 252 and/or the second guide roll 332 rotate, the transfer member 338 advances in a direction indicated by arrow A. The continuous barrier substrate 242 may be disposed on the external surface of the transfer member 338 such that as the transfer member 338 advances, the continuous barrier substrate 242 may advance in the machine direction MD toward the folding roll 266.

At least one of the first guide roll 252 and the second guide roll 332 may be driven by a motor. The motor may be any device that transmits rotational energy to the guide roll. The motor may be operatively linked or operatively engaged with the guide roll using any technique known to those skilled in the art such as, for example, a gear to gear connection, transmission belting and pulleys, gearboxes, direct couplings, and the like or any combination thereof. The motor may be configured to rotate at least one of the first guide roll 252 and the second guide roll 332 at a variable angular velocity or a constant velocity. The variation in rotation of at least one of the first guide roll 252 and the second guide roll 332 may result in the variable advancement of the transfer member 338. Similarly, the constant rotation of the first guide roll 252 and the second guide roll 332 may result in the continuous advancement of the transfer member 338. For example, in some embodiments, the continuous barrier substrate 242 may be advanced in the machine direction MD by the infeed device 251 such that the continuous barrier substrate 242 may be supplied at a constant first velocity V₁.

The continuous barrier substrate 242 may be advanced toward a folding roll 266, as shown in FIG. 5A. The folding roll 266 may be configured to fold a portion of the continuous barrier substrate 242. The folding roll 266, such as disclosed, for example, in U.S. Patent Application No. 62/004,240 filed May 29, 2014, may include a folding member 342. The folding member 342 may include any number of grooves, such as a first groove, a second groove, and a third groove, also referred herein as folds. The folding member 342 may be removably associated with the folding roll 266 or, alternatively, the folding roll 266 may be machined such that a portion of the folding roll includes one or more grooves. The folding roll 266 may be configured to rotate about an axis of rotation 267. The folding roll 266 may be driven such that the folding roll 266 rotates at a constant second velocity V₂ about the axis of rotation 267. The second velocity V₂ may be substantially equal to the velocity at which the first cuff substrate 204 and the second cuff substrate 206 are advanced to the first bonding area 276.

The velocity at which the continuous barrier substrate 242 advances toward the folding roll 266 and the second velocity V₂ may determine, in part, the diameter D of the folding roll 266 and the number of folding members 242 and/or groove portions 272 positioned about the outer circumferential surface 270 of the folding roll 266. For example, in some embodiments, the diameter D of the folding roll 266 and the number of folding members 242 and/or groove portions 272 may be such that outer circumferential surface 270 is two times a product pitch. A product pitch may be defined as the length from the beginning to the end of one product, as illustrated in FIGS. 2 and 4 as the length L. Thus, the folding roll 266 may include two folding members 242 and/or groove portions 272 spaced equidistant about the outer circumferential surface 270 of the folding roll 266. The folding roll 266 may be designed such that at least one discrete barrier member is included in each final product and each barrier member includes one or more rugosities formed by the folding roll 266.

As illustrated in FIG. 5A, folding roll 266 may include an outer circumferential surface 270. The continuous barrier substrate 242 may be advanced from the infeed device 251 onto the outer circumferential surface 270 of the folding roll 266. To aid in the transfer of the continuous barrier substrate 242 onto the folding roll 266, the folding roll may be operatively engaged with a vacuum source configured to apply a vacuum force to one or more portions of the continuous barrier substrate 242, and the folding roll may include one or more portions configured to engage with the continuous barrier substrate 242. More specifically, the folding roll 266 may include a first engagement portion 271, a second engagement portion 273 opposite the first engagement portion, and a groove portion 272 between the first engagement portion 271 and the second engagement portion 237. It is to be appreciated that the folding roll 266 may include any number of each of the first engagement portion 271, the second engagement portion 372, and groove portion 272. As previously stated, the groove portion 272 may include any number of folds, also referred herein as grooves. For example, a groove portion may include any number of peak regions and valley regions making up one or more folds or grooves. Each fold or groove may have various profiles, such as semi-circular, square, rectangular, and the like. Further, each groove or fold may be linear, curvilinear, or any other shape that would act as a barrier for bodily exudates in the absorbent article. The groove profile may vary in so far as the profile aids in allowing the substrate to be drawn into and conform to the profile via vacuum force. It is to be appreciated that the final profile or shape of the substrate may or may not be the same as the profile or shape of the groove.

As previously stated, the folding roll 266 may be in fluid communication with a vacuum source (not shown) which may move fluid, such as air, from adjacent the surface of the folding roll toward the axis of rotation 267 to provide a vacuum force F. The vacuum force F may act on the continuous barrier substrate 242 such that a first surface 241 of the continuous barrier substrate 242 associates with the outer surface 270 of the folding roll 266 and the continuous barrier substrate 242 may remain associated with the folding roll 266 during rotation. More specifically, the infeed device 251 may cause the continuous barrier substrate 242 to be advanced onto the outer circumferential surface 270 of the folding roll 266, as illustrated in FIG. 5A. The continuous barrier substrate 242 may be advanced by the infeed device 251 at a first velocity V₁, which is less than the second velocity V₂, the velocity at which the folding roll 266 rotates about the axis of rotation 267. The amount of the continuous barrier substrate 242 that is advanced onto the outer circumferential surface 270 of the folding roll 266 may depend, in part, on the difference between the first velocity V₁ and the second velocity V₂. In the instance that the second velocity is greater than the first velocity, the continuous barrier substrate may be disposed on the outer circumferential surface 270 at a slower rate than the rate at which the folding roll rotates. Thus, the continuous barrier substrate 242 slips on the outer circumferential surface 270 of the folding roll 266.

The continuous barrier substrate 242 may continue to be disposed on and slip along the outer circumferential surface 270 until the first engagement portion 271 associates with the beginning edge portion 344 of the continuous barrier substrate 242. As the folding roll 266 rotates about the axis of rotation 267, the first engagement portion 271 engages the beginning edge portion 344 of the continuous barrier substrate 242. More specifically, the first engagement portion 271 may be fluidly connected to the vacuum source. As the first engagement portion 271 engages the beginning edge portion 344, the vacuum source provides a vacuum force F on the beginning edge portion 344 of the continuous barrier substrate 242 such that the beginning edge portion 344 is engaged with the first engagement portion 271 and begins to rotates with the folding roll 266.

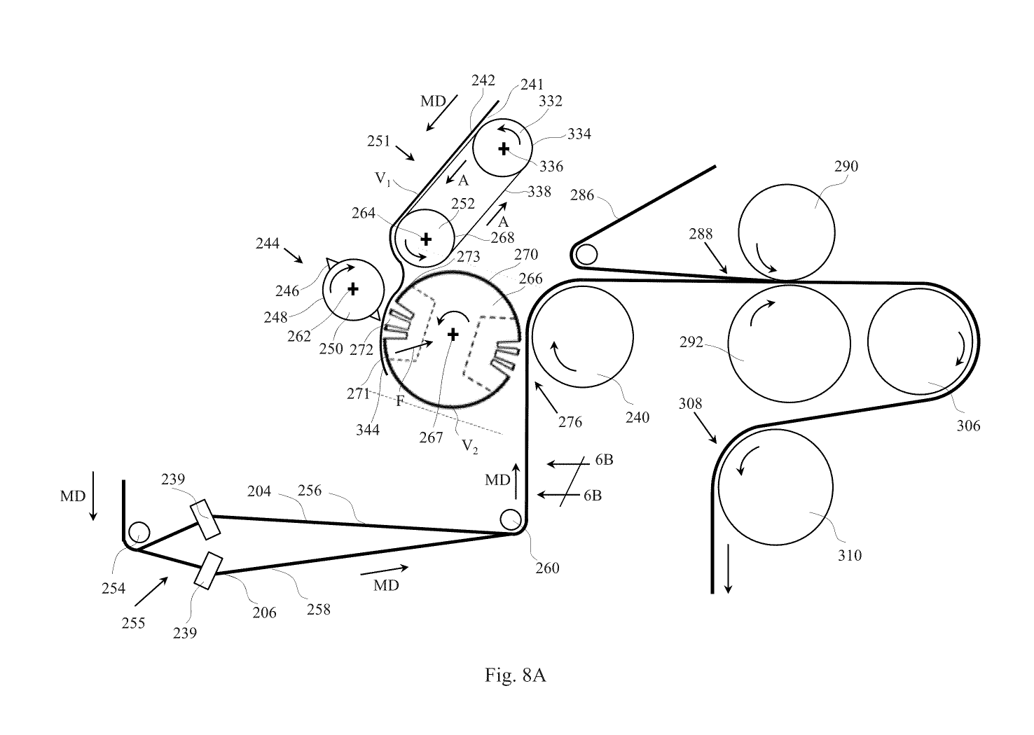

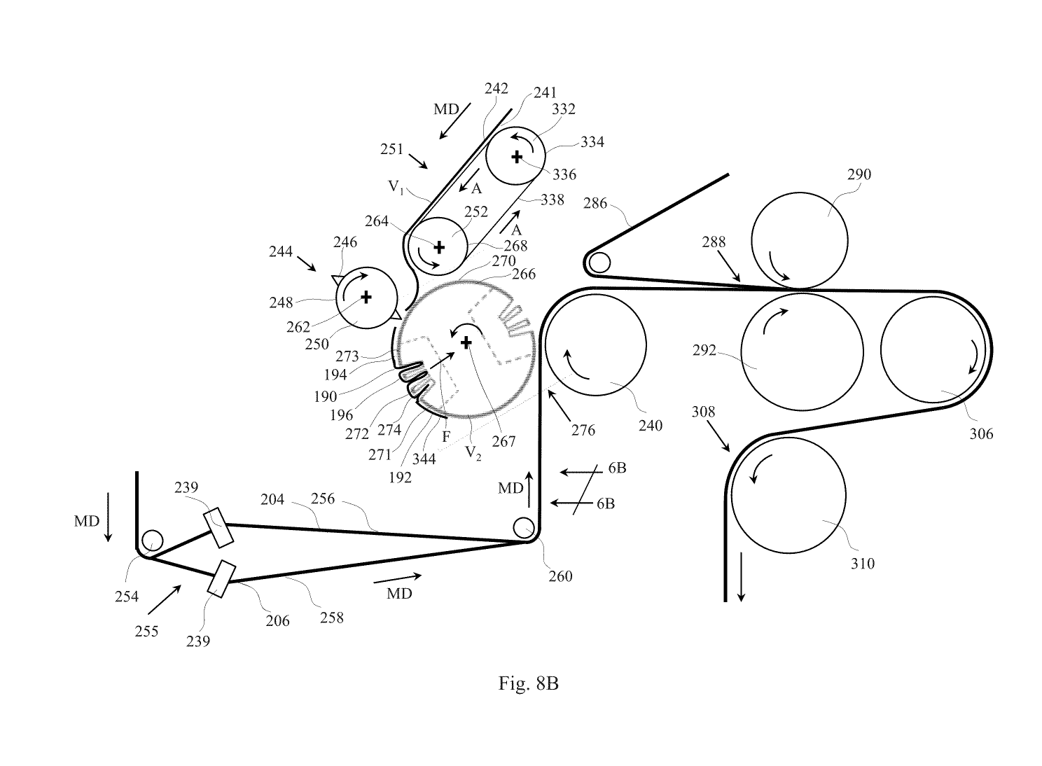

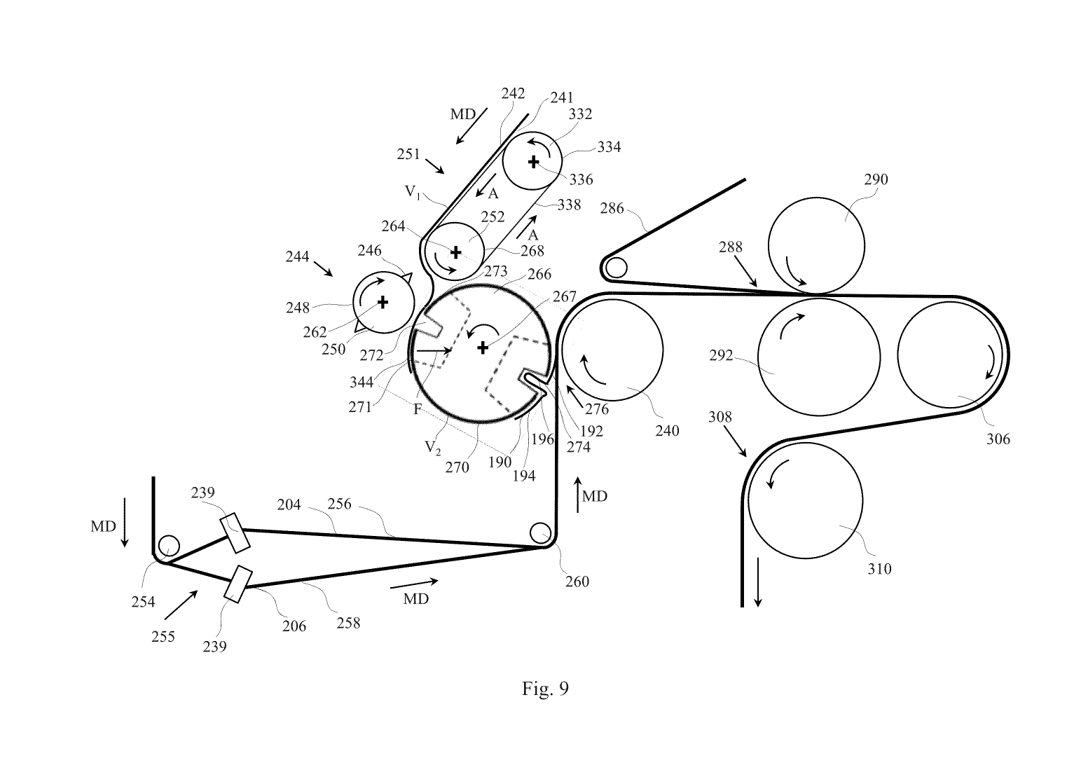

As illustrated in FIG. 8A, a cutting device 244 may be positioned adjacent the outer circumferential surface 270 of the folding roll 266. In some example embodiments, the cutting device 244 may include a cutting roll 250 that operatively engages the folding roll 266. The cutting roll 250 may include a blade 246 that extends radially outward from the surface 248 of the cutting roll 250. The cutting roll 250 may rotate about a central axis 262 causing the blades 246 to rotate about the central axis 262. The blade 246 may engage the continuous barrier substrate 242 separating a portion of the barrier substrate 242 to form a discrete barrier member 190. Thus, as the continuous barrier substrate 242 is engaged by the first engagement portion 271 and a portion of the continuous barrier substrate is disposed across the groove portion 272 and the second engagement portion 273, the cutting device 244 may sever, also referred here as separate, a portion of the continuous barrier substrate 242 to form a discrete barrier member 190, as illustrated in FIG. 8B.

It is to be appreciated that the continuous barrier substrate 242 is merely disposed on the groove portion 272 and does not fill the grooves or folds of the groove portion. It is also to be appreciated that the cutting device 242 may sever the portion of the continuous barrier substrate 242 that is disposed on the second engagement portion 273 or any portion of the continuous barrier substrate 242 disposed on the outer circumferential surface 270 that is positioned after, in the machine direction, the second engagement portion 273. The location at which the continuous barrier substrate 242 is severed may depend, at least in part, on the number of grooves in the groove portion 272, the speed at which the continuous barrier substrate 242 is fed onto the folding roll 266, and the speed at which the folding roll 266 rotates about the axis of rotation 267.

Subsequent to the vacuum force F acting on the first engagement portion 271 and the cutting device 244 severing a portion of the continuous barrier substrate 242 to form a discrete substrate, the vacuum source may be configured to apply a vacuum force on one or more portions of the discrete barrier member 190. More specifically, a second vacuum force may be supplied by the same vacuum source or another vacuum source such that a portion of the discrete barrier member 190 may be displaced into the grooves, also referred to as folds, of the groove portion 272, as illustrated in FIG. 8B. This forms the one or more rugosities in the barrier substrate 190. Further, a third vacuum force may be supplied by the same vacuum source or another vacuum source such that the portion of the continuous barrier substrate 242 disposed on the second engagement portion 273 may be held to the second engagement portion 273 as the folding roll 266 rotates.

The one or more vacuum sources may be controlled such that the vacuum force F is applied sequentially to the discrete barrier substrate 242. For example, in some embodiments, the vacuum force F may be applied first to the portion of the continuous barrier substrate 242 in the first engagement portion 271, then to the portion of discrete barrier substrate 190 disposed on the groove portion 272 and, finally, to the portion of the discrete barrier substrate 190 disposed on the second engagement portion 273 and/or the outer circumferential surface 270 following the groove portion. The controlled, sequential application of the vacuum force F allows a sufficient amount of the continuous barrier substrate 272 to be advanced by the infeed device 251 such that the beginning edge portion 344 remains substantially disposed on the first engagement portion 271 while a sufficient amount of the barrier substrate is disposed on the groove portion 272 such that the discrete barrier substrate 190 can be forced into the grooves or folds and the portion of the discrete barrier substrate 190 remaining may be disposed on the second engagement portion 273. A portion of the discrete barrier substrate 190 may fill the grooves of the groove portion 272 while not displacing the portion of the continuous barrier substrate 242 disposed on the first engagement portion 271. Similarly, a portion of the discrete barrier substrate may be disposed on the second engagement region 273 while not displacing the portion of the discrete barrier substrate 190 disposed on the first engagement portion 271 and filling the groove(s) or fold(s) of the groove portion 272.

It is also to be appreciated that the vacuum source may supply a continuous vacuum force across each of the first engagement portion 271, the groove portion 272, and the second engagement portion 273. However, the vacuum force F may vary in the amount of force applied to the continuous substrate assembly 242. For example, a first vacuum force may be applied to the first engagement portion 271, a second vacuum force may be applied to the groove portion 272, and a third vacuum force may be applied to the second engagement portion 273. Each of the first vacuum force, the second vacuum force, and the third vacuum force may be exerted on the barrier substrate. However, prior to the cutting device 244 severing the continuous barrier substrate 242, the second vacuum force and the third vacuum force may not be strong enough to cause the barrier substrate to be displaced and/or held in a fixed position. However, the second vacuum force and the third vacuum force may be strong enough to aid in the transfer of the continuous barrier substrate 242 from the transfer member 338 to the folding roll 266. Once the continuous barrier substrate 242 has been severed, the second vacuum force and the third vacuum force may increase accordingly to cause the discrete barrier substrate 190 to fill the one or more grooves and to be held in position on the second engagement portion. It is also to be appreciated that the vacuum force may be applied non-uniformly across the discrete barrier substrate 190. For example, in some embodiments, the first vacuum force may be greater than the second vacuum force and the third vacuum force. Further, in some embodiments, the first vacuum force may be greater than the second vacuum force, and the second vacuum force may be greater than the third vacuum force. Further still, the second vacuum force may be greater than at least one of the first vacuum force and the third vacuum force.

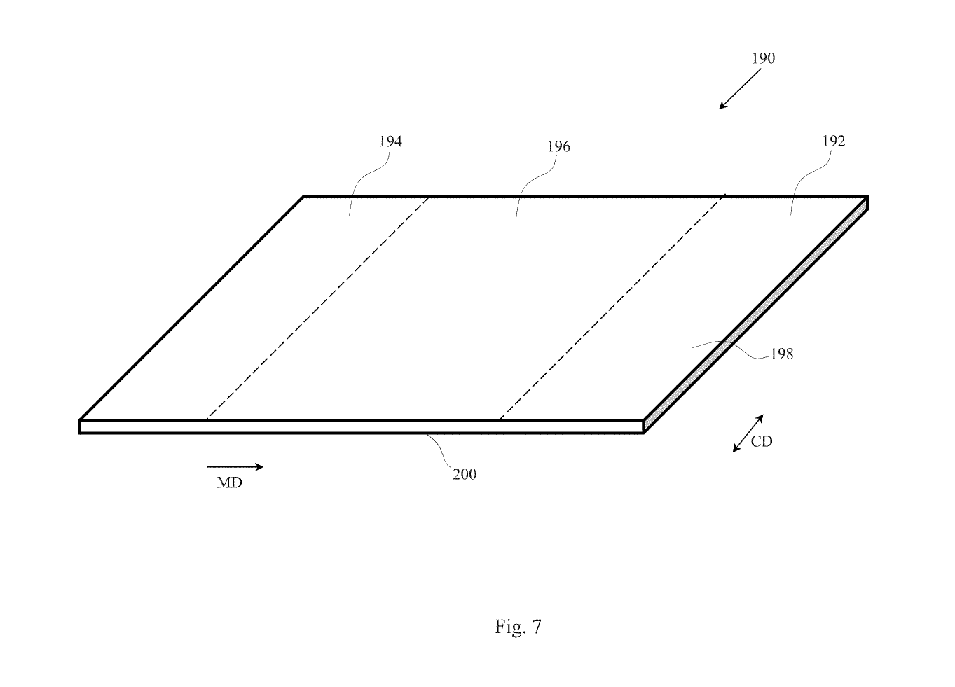

The discrete barrier member 190 may include a leading edge portion 192, a trailing edge portion 194 opposite the leading edge portion, and a central portion 196 between the leading edge portion 192 and the trailing edge portion 194, as shown in FIGS. 7 and 8B. The discrete barrier member 190 may also include a first surface 198 and a second surface 200 opposite the first surface 198. The discrete barrier member 190 may be extensible in at least one of the machine direction and the cross direction.

The discrete barrier substrate 190 may continue to be positioned on the outer surface 270 of the folding roll 266, as illustrated in FIG. 8B, such that the leading edge portion 192 may associate with the first engagement portion 271 of the folding roll 266. Similarly, the trailing edge portion 194 may associate with the second engagement portion 273 of the folding roll 266. The central portion 196 of the discrete barrier member 190 may associate with the groove portion 272. The vacuum force F may hold the central portion 196 of the discrete barrier member 190 in the groove portion 272 holding the formation of the fold 274 in the discrete barrier member 190. The discrete barrier member 190 may be advanced toward the first bond roll 240, the first cuff substrate 204, and the second cuff substrate 206 at the second velocity.

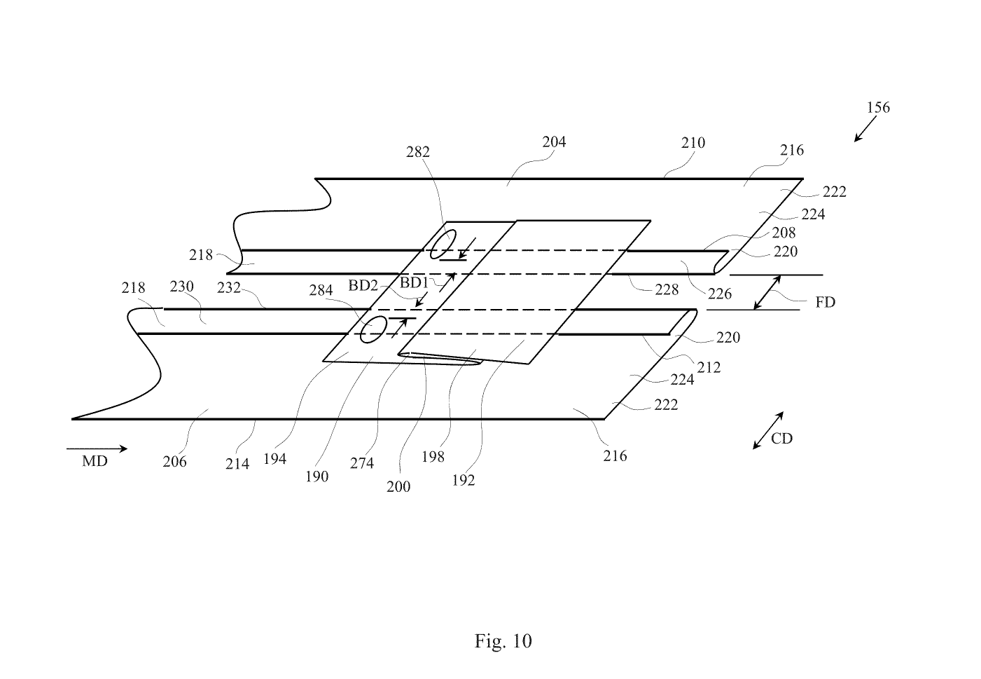

As previously stated, it is to be appreciated that the folding roll 266 may include a groove portion 272. The groove portion 272 may include one or more grooves, also referred to herein as folds. For ease of explanation, FIG. 9 illustrates a folding roll 266 including groove portions 272 each having one groove. It is also to be appreciated that different groove portions may include the same or different numbers of grooves. For example, a first groove portion may include one groove and a second groove portion may include three grooves.

Referring to FIG. 9, the leading edge portion 192 of the discrete barrier member 190 may first associate with the first cuff substrate 204 and the second cuff substrate 206 as the folding roll 266 rotates. The discrete barrier member 190 associated with the first cuff substrate 204 and the second cuff substrate 206 may advance into a first bonding area 276. The first bonding area 276 may include the first bond roll 240. The first bond roll 240 may interact with the folding roll 266 to form a bond. In some embodiments, the first bond roll 240 may be an anvil roll including one or more bond patterns. In the first bonding area 276, at least a portion of the trailing edge portion 194 of the discrete barrier member 190 may be connected to the first cuff substrate 204 and the second cuff substrate 206. In some embodiments, the trailing edge portion 194 of the discrete barrier member 190 may be bonded to at least a portion of the first cuff fold 226 of the first cuff substrate 204 and to the second cuff fold 230 of the second cuff substrate 206. As the discrete barrier member 190 is transferred from the folding roll 266, the vacuum force F provided by the folding roll 266 may be removed.

The portion of the trailing edge portion 194 of the discrete barrier member 190 may be bonded at a first bond site 282 and a second bond site 284, as illustrated in FIG. 10. The bond between the discrete barrier member 190 and the first and second cuff substrate 204, 206 may be by, for example, high pressure welding, hot air welding, heat crimping, or ultrasonic welding. Exemplary bonding methods and apparatuses may include those described in U.S. Pat. Nos. 4,854,984; 4,919,738; 5,711,847; 5,817,199; 6,123,792; 7,449,084; 6,248,195; 6,546,987; and U.S. patent application Ser. No. 14/038,812; 61/836,690; and 61/836,745. It is to be appreciated that the bond between the discrete barrier member 190 and the first and second cuff substrates 204, 206 may also include the use of adhesives alone or in addition to the aforementioned types of bonding. However, it has been found that limiting the use of adhesives in absorbent articles, such as diapers, is desirable to consumers and manufacturers. For consumers, the desire for limited use or non-use of adhesives may be a result of, for example, actual or perceived irritation of the wearer's skin. For manufacturers, the desire for limited use of adhesive may be a result of numerous challenges in handling the adhesive during the manufacturing process. For example, adhesives often require a certain period of time to adhere and/or solidify, which may cause a delay in the manufacturing process. Although adhesives may be used in absorbent articles, the absorbent article including a discrete barrier member of the present disclosure may be assembled without the use of adhesives. The bond formed at the first bond site 282 and the second bond site 284 may be strong enough to withstand a greater than 105% and/or greater than 125% and/or greater than 200% and/or greater than 250% elongation in the width of the discrete barrier member 190.

Further, the first bond site 282 may be positioned at a first bond distance BD1 from the first fold edge 228. The first bond distance BD1 is the substantially perpendicular distance from the first fold edge 228 to the first bond site 282. For example, the first bond distance BD1 may be from about 1 mm to about 20 mm and/or from about 2 mm to about 10 mm and/or from about 3 mm to about 5 mm, including all 0.5 mm increments therebetween. Likewise, the second bond site 284 may be posited a second bond distance BD2 from the second fold edge 232. The second bond distance BD2 is the substantially perpendicular distance form the second fold edge 232 to the second bond site 284. For example, the second bond distance BD2 may be from about 1 mm to about 20 mm and/or from about 2 mm to about 10 mm and/or from about 3 mm to about 5 mm, including all 0.5 mm increments therebetween. The second bond distance BD2 may be greater than, less than, or equal to the first bond distance BD1 It is to be appreciated that the bond distance may be measured from the first inner cuff edge 208 and the second inner cuff edge 212 if the first cuff substrate 204 and the second cuff substrate 206 are not folded.

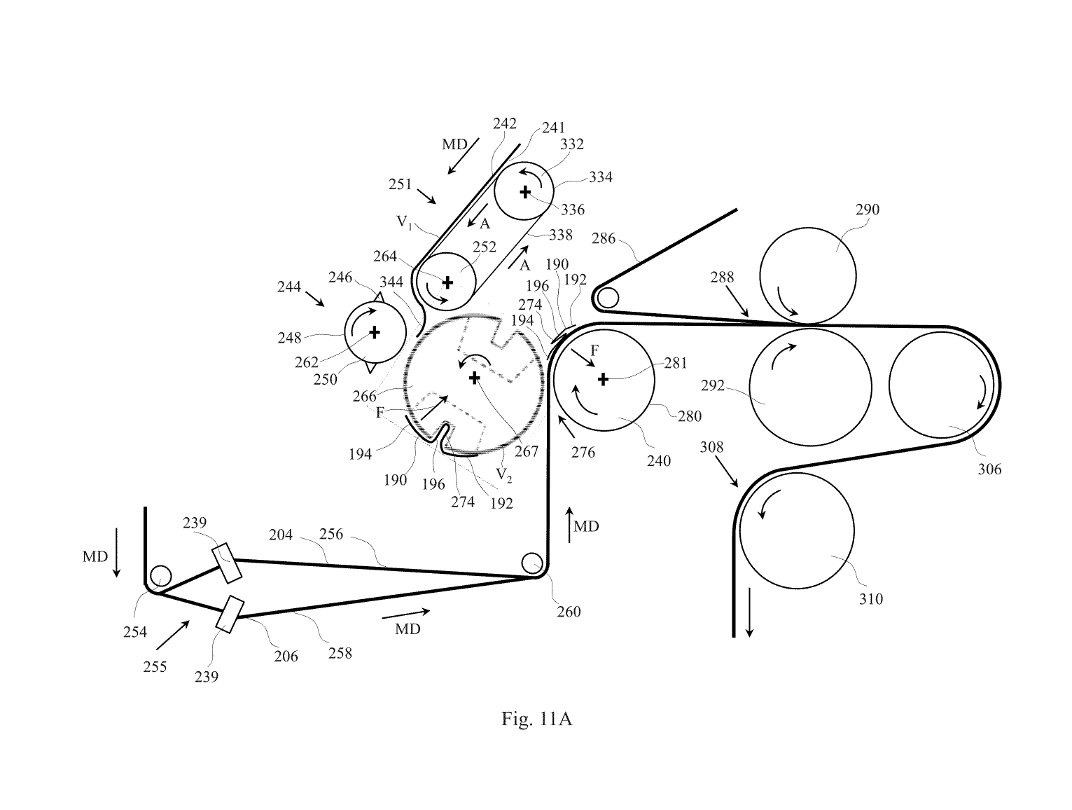

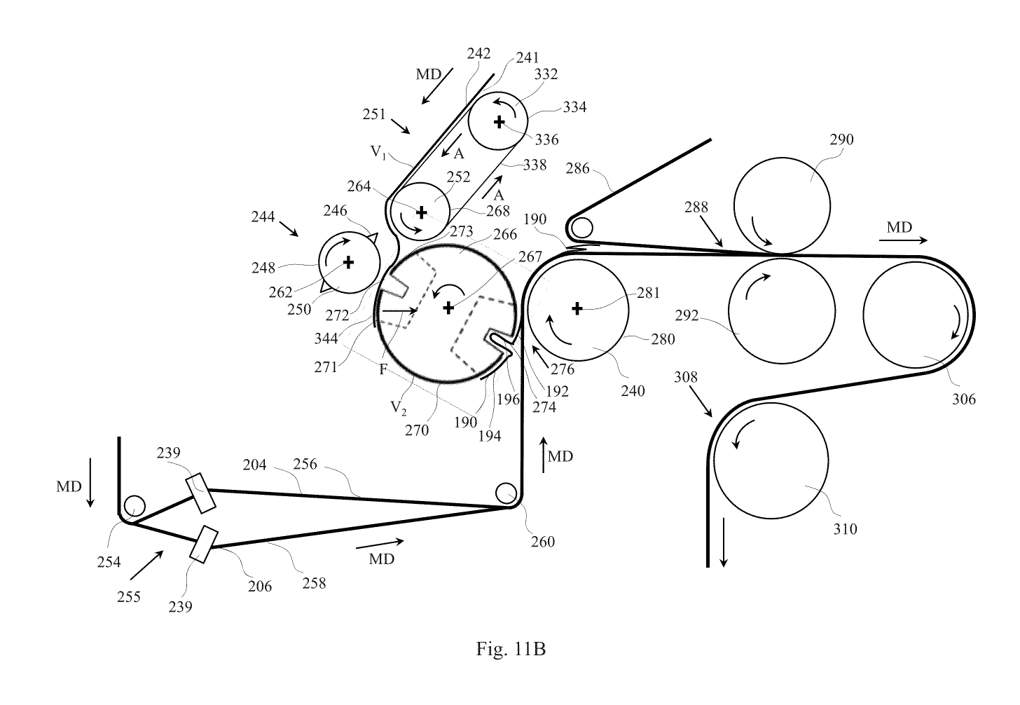

The trailing edge portion 194 may associate with the first cuff substrate 204 and the second cuff substrate 206, as illustrated in FIG. 11A. The first cuff substrate 204, the second cuff substrate 206, and the discrete barrier member 190 may advance on the outer surface 280 of the bond roll 240. The bond roll may rotate about an axis of rotation 281. Further, the bond roll 240 may be in fluid communication with a vacuum source (not shown). The vacuum source provides a vacuum force F on the discrete barrier member 190, the first cuff substrate 204, and the second cuff substrate 206 such that each maintains association with the outer surface 280 of the bond roll 240, as illustrated in FIG. 11A. More specifically, the second surface 200 of the discrete barrier member 190 may be associated with the first cuff substrate 204 and the second cuff substrate 206. The fold 274 formed by the folding roll 266 may be pulled to the surface 280 of the bond roll 240 by the vacuum force F. Thus, the fold 274 may be substantially planar with the leading edge portion 192 and the trailing edge portion 194, as illustrated in FIG. 10. The vacuum force F may hold the fold 274 and the leading edge portion 192 of the discrete barrier member 190 in position as the first cuff substrate 204, the second cuff substrate 206, and the discrete barrier member 190 are advanced to a topsheet substrate 286.

It is to be appreciated that in some embodiments, prior to the topsheet 286 being disposed on the first cuff substrate and the second cuff substrate, the discrete barrier member 190, the first cuff substrate 204, and the second cuff substrate may be advanced onto a vacuum roll (not shown). The vacuum roll may be in fluid communication with a vacuum source. The vacuum source provides a vacuum force F on the discrete barrier member 190, the first cuff substrate 204, and the second cuff substrate 206 such that each maintains association with the outer surface of the vacuum roll as the first cuff substrate 204, the second cuff substrate 206, and the discrete barrier member 190 are advanced to the topsheet substrate 286.

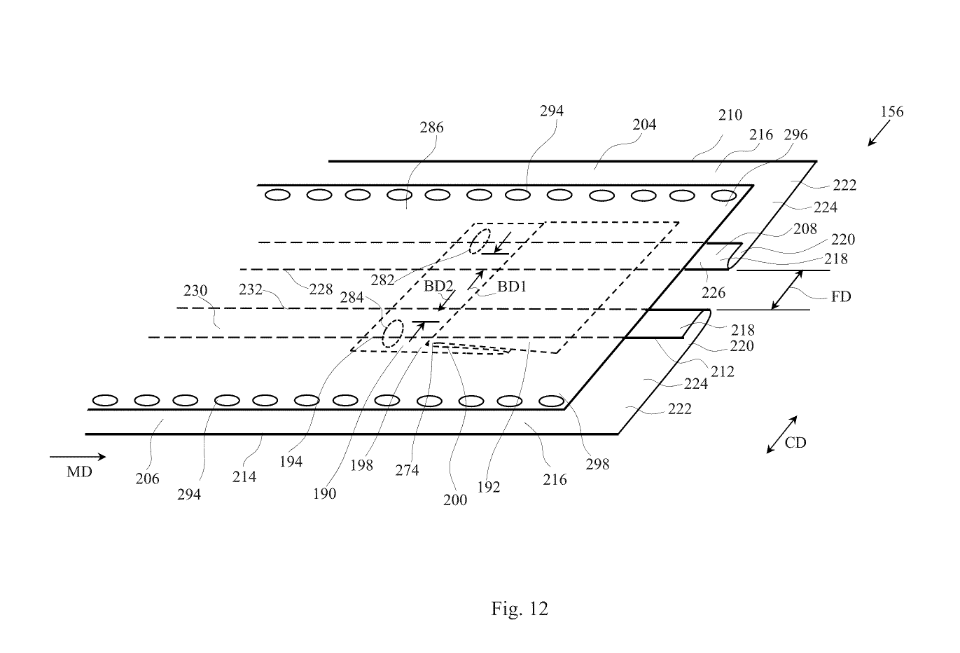

As illustrated in FIGS. 11B and 12, a topsheet substrate 286 may be advanced in the machine direction and may be disposed on at least a portion of the first surface 198 of the discrete barrier member 190. In some exemplary embodiments, the topsheet substrate 286 may substantially cover the discrete barrier member 190. Further, the topsheet substrate 286 may be disposed on at least a portion of the first cuff substrate 204 and the second cuff substrate 206. Thus, the discrete barrier member 190 may be positioned between the topsheet substrate 286 and the first and second cuff substrates 204, 206. The topsheet substrate 286 disposed on the discrete barrier member 190 and at least a portion of the first cuff substrate 204 and the second cuff substrate 206 may be advanced to a second bonding area 288. The second boding area 288 may connect the topsheet substrate 286 to at least a portion of the first cuff substrate 204 and the second cuff substrate 206. The second bonding area 288 may include a second bond roll 290 and a third bond roll 292. The second bond roll 290 may operatively engage the third bond roll 292 to form a linear bond 294 between the topsheet substrate 286 and the first cuff substrate 204 and the topsheet substrate 286 and the second cuff substrate 206, as illustrated in FIG. 12. More specifically, the topsheet substrate 286 may be bonded to at least one of the first cuff surface 216 and the second cuff surface 218 of each of the first cuff substrate 204 and the second cuff substrate 206. For example, in some embodiments, the topsheet substrate 286 may be bonded to the first cuff surface 216 of each of the first cuff substrate 204 and the second cuff substrate 206. The linear bond 294 may extend longitudinally in the machine direction MD on at least one of the first cuff surface 216 and the second cuff surface 218 of each of the first cuff substrate 204 and the second cuff substrate 206, as illustrated in FIG. 12. The bond may be made by, for example, high pressure welding, hot air welding, heat crimping, or ultrasonic welding. Exemplary bonding methods and apparatuses may include those described in U.S. Pat. Nos. 4,854,984; 4,919,738; 5,711,847; 5,817,199; 6,123,792; 7,449,084; 6,248,195; 6,546,987; and U.S. patent application Ser. No. 14/038,812; 61/836,690; and 61/836,745.

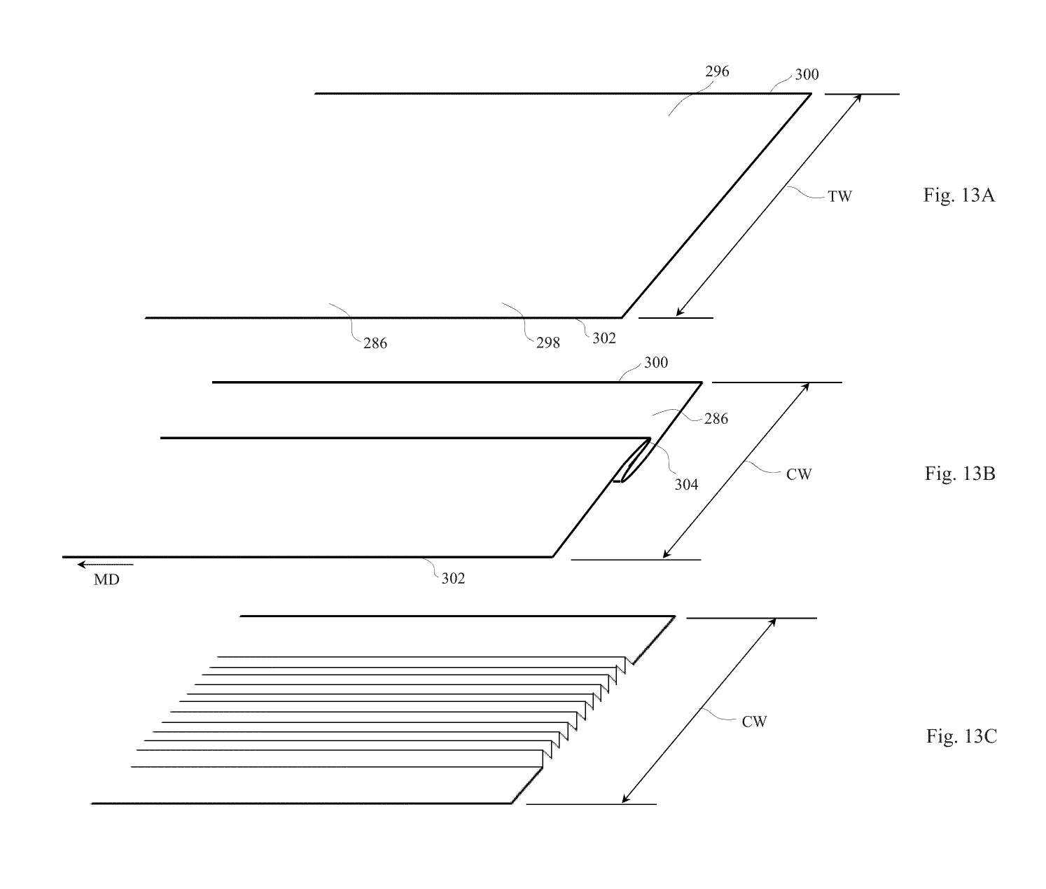

As previously discussed, the first cuff substrate 204 may be a first distance FD away from the second cuff substrate 206. Having the cuff separated by a first distance may allow the discrete barrier member 190 to be bonded to the first cuff substrate 204 and the second cuff substrate 206 in a relaxed or unstretched state. However, in some embodiments, having the first cuff substrate 204 being separated by a first distance FD from the second cuff substrate 206 when the topsheet substrate 286 is disposed on the discrete barrier member 190, the first cuff substrate 204, and the second cuff substrate 206 may create a problem. More specifically, the topsheet substrate 286 may have a topsheet width TW, a first topsheet edge region 296, a second topsheet edge region 298 opposite the first topsheet edge region 298, a first topsheet edge 300, and a second topsheet edge opposite the first topsheet edge, as illustrated in FIG. 13A. If the first cuff substrate 204 and the second cuff substrate are separated by a first distance FD, the topsheet width TW may position each of the first topsheet edge region 296 and the second topsheet edge region 298 too far away from the first fold edge 228 and the second fold edge 232, respectively, for the linear bond 294 to be positioned in the desired location. Stated another way, in some exemplary embodiments, the topsheet substrate 286 may be positioned with respect to the first cuff substrate 204 such that at least one of the first fold edge 230 and the first inner cuff edge 208 are at a distance from about 15 mm to about 45 mm and/or about 20 mm to about 35 mm and/or about 25 mm to about 32 mm to the first topsheet edge 300. Similarly, the topsheet substrate 286 may be positioned with respect to the second cuff substrate 206 such that at least one of the second fold edge 232 and the second inner cuff edge 212 are at a distance from about 15 mm to about 45 mm and/or about 20 mm to about 35 mm and/or about 25 mm to about 32 mm to the second topsheet edge 302.

Therefore, when the position of the first cuff substrate 204 and the second cuff substrate 206 do not allow the topsheet substrate 286 to be disposed in the proper position for bonding, the topsheet substrate 286 may be manipulated prior to being disposed on the first cuff substrate 204, the second cuff substrate 206, and the discrete barrier member 190. For instance, the topsheet substrate 286 may be manipulated to include a topsheet fold 304, as illustrate in FIG. 13B. The topsheet fold 304 may be substantially parallel to at least one of the first topsheet edge 300 and the second topsheet edge 302. The topsheet fold 304 may extend in the machine direction MD. The topsheet fold 304 may form a compressed topsheet width CW that is less than the topsheet width TW. It is to be appreciated that the number of topsheet folds 304 and/or the width of the topsheet fold may depend on the first distance FD between the first cuff substrate 204 and the second cuff substrate 206.

In some exemplary embodiments, the topsheet substrate 286 may undergo mechanical activation to form a topsheet substrate having a compressed topsheet width CW and to impart extensibility in at least one of the lateral direction and the longitudinal direction of the topsheet substrate 286. Exemplary mechanical activation methods and apparatuses may include those described in U.S. Pat. Nos. 6,632,504; 5,916,661; 5,628,097, and U.S. Patent Publication No. 2003/0021651, and U.S. patent application Ser. Nos. 14/032,595; 14/247,276; 14/270,468. The mechanical activation of the topsheet substrate 286 may result in the topsheet substrate 286 having a compressed topsheet width CW that is less than the topsheet width TW, as illustrated in FIG. 13C. It is to be appreciated that the type and amount of mechanical activation may depend on the first distance FD between the first cuff substrate 204 and the second cuff substrate 206. It is also to be appreciated that any number of methods to reduce the width of the topsheet substrate may to be used so that the topsheet substrate 286 may be appropriately positioned on the first cuff substrate 204 and the second cuff substrate 206. For example, a topsheet substrate 286 may undergo folding and mechanical activation to reduce to topsheet width TW to the compressed topsheet width CW.

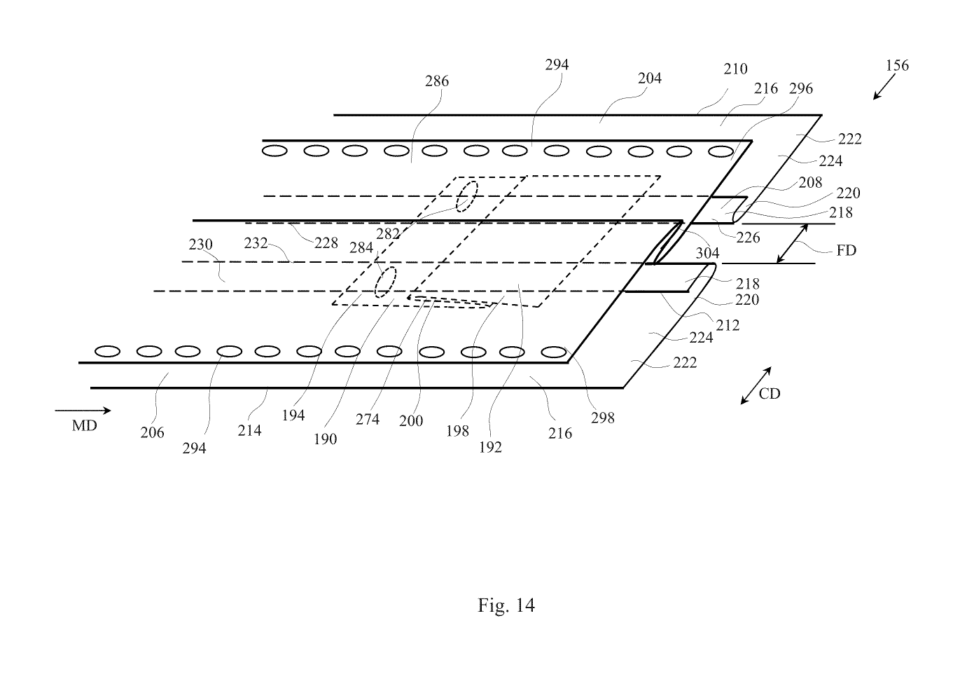

FIG. 14 illustrates a topsheet substrate 286 having a compressed topsheet width CW disposed on the first cuff substrate 204, the second cuff substrate 206, and the discrete barrier member 190. The compressed topsheet width CW may allow the topsheet substrate 286 to be disposed on the first cuff substrate 204 and the second cuff substrate 206 such that the topsheet substrate 286 may be in the desired position to be connected to the first and second cuff substrates 204, 206.

It is to be appreciated that the first cuff substrate 204 and the second cuff substrate 206 may be separated by a distance such that the topsheet width TW does not need to be reduced to the compressed topsheet width CW, and the topsheet substrate 286 can be disposed on the first cuff substrate 204 and the second cuff substrate 206 without any prior manipulation, such as folding, as illustrated in FIG. 12. Assuming that the first cuff substrate 204 and the second cuff substrate 206 are separated by a distance that allows for the topsheet substrate 286 to be positioned without prior manipulation, the topsheet substrate 286 may be advanced to a fourth bonding roll 310. At the fourth bonding roll 310, the topsheet substrate 286 may be bonded to the discrete barrier member 190, the first cuff substrate 204, and the second cuff substrate 206, as will be discussed in more detail below.

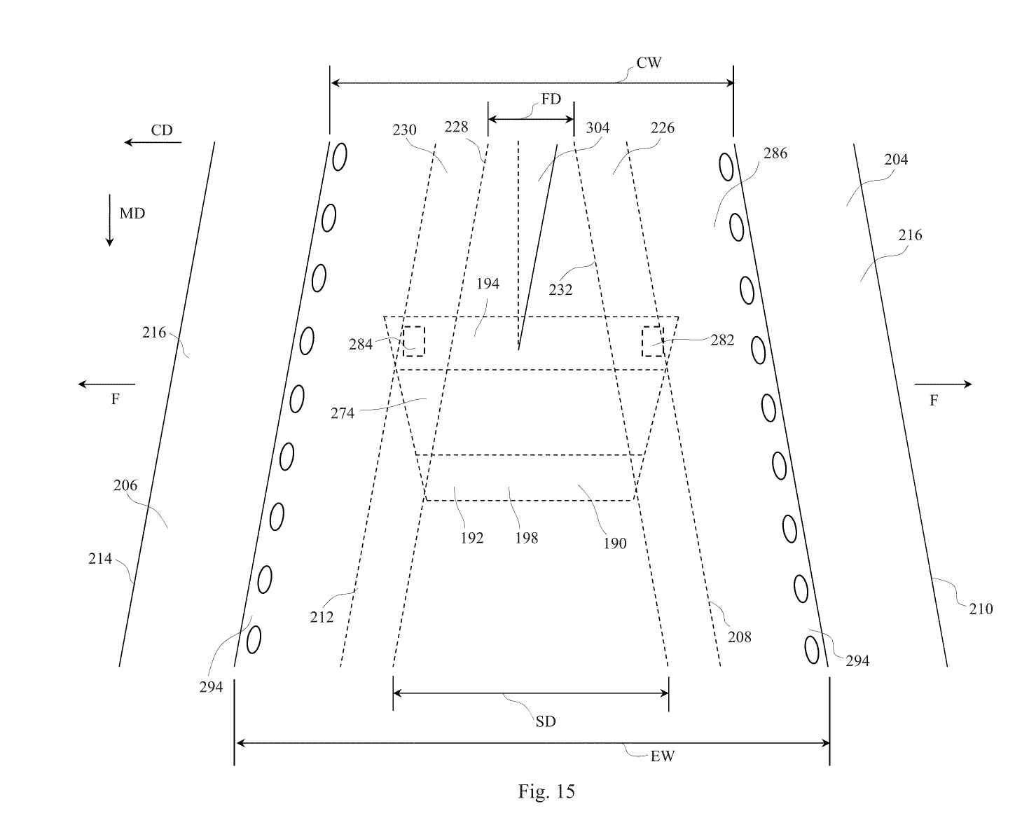

Alternatively, assuming that the first cuff substrate 204 and the second cuff substrate 206 are separated by a first distance FD and the topsheet substrate 286 is disposed on the first cuff substrate 204 and the second cuff substrate 206 with a compressed topsheet width CW, as illustrated in FIG. 14, this assembly may be advanced to a separation roll 306, as illustrated in FIG. 11B. The separation roll 306 may separate the first cuff substrate 204 from the second cuff substrate 206 and extend, also referring to herein as stretch, the topsheet substrate 286 and the discrete barrier member 190, as illustrated in FIG. 15. A force F may be applied to the first cuff substrate 204 and the second cuff substrate 206 to separate the first cuff substrate 204 from the second cuff substrate 206 and to extend the topsheet substrate 286 and the discrete barrier member 190. More specifically, the force F acts in a direction substantially perpendicular to the machine direction MD, as shown in FIG. 15. During the time the force F acts on each cuff substrate, the linear bond 294 between each of the first cuff substrate 204 and the second cuff substrate 206 and the topsheet substrate 286 continues to connect the topsheet substrate 286 to each cuff substrate 204, 206. Similarly, the bond between the trailing edge portion 194 and the first and second cuff substrates 204, 206 also remains connected during the separation of the cuff substrates. Thus, in some embodiments, the linear bond 294 may be strong enough to withstand a greater than 60% and/or greater than 75% and/or greater than 100% and/or greater than 130% elongation in the width of the topsheet substrate 286. The first bond site 282 and the second bond site 284 may be strong enough to withstand a greater than 105% and/or greater than 125% and/or greater than 200% and/or greater than 250% elongation in the width, parallel to the cross direction, of the discrete barrier member 190.

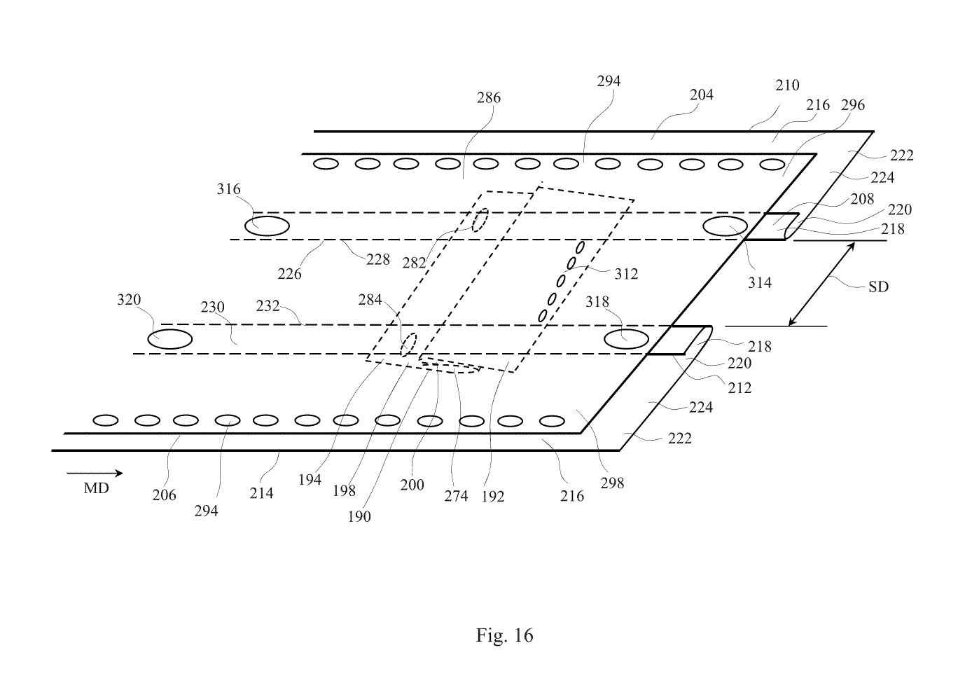

Once fully extended, the first cuff substrate 204 and the second cuff substrate 206 may be separated by a second distance SD, which may be greater than the first distance FD, as illustrated in FIGS. 15 and 16. More specifically, the first fold edge 228 may be separated from a second fold edge 232 by the second distance SD. The second distance SD may be from about 110 mm to about 45 mm and/or from about 96 mm to about 55 mm and/or from about 80 mm to about 64 mm, including all 0.5 mm therebetween. The topsheet substrate 286 may also be extended. The topsheet substrate 286 may be extended by an amount about equal to the second distance SD. Thus, the topsheet substrate 286 may have an extended topsheet width EW in the cross direction between the first topsheet edge and the second topsheet edge. The extended topsheet width EW may be equal to about the compressed topsheet width added to the second distance SD minus the first distance FD, or the distance the first cuff substrate 204 was separated from the second cuff substrate 206. In some embodiments, the extended topsheet width EW may be equal to about the topsheet width TW.

Referring to FIG. 11B, the extended topsheet substrate 286, first cuff substrate 204, and second cuff substrate 206 may be advanced to a third bonding area 308. The third bonding area 308 may comprise a fourth bond roll 310. The fourth bond roll 310 may connect the topsheet substrate 286 to the discrete barrier member 190, the first cuff substrate 204, and the second cuff substrate, as illustrated in FIG. 16. More specifically, at least a portion of the leading edge portion 192 of the discrete barrier member 190 may be bonded to a portion of the topsheet substrate 286. The bond between the discrete barrier member 190 and the topsheet substrate 286 may be a linear bond 312. The fourth bond roll 310 may also bond the topsheet substrate 286 to the first cuff substrate 204 and the second cuff substrate 206. The topsheet substrate 286 may be bonded to the first cuff substrate 204 at a first bond area 314 and a second bond area 316. Similarly, the topsheet substrate 286 may be bonded to the second cuff substrate 206 at a third bond area 318 and a fourth bond area 320. The bond between the topsheet substrate 286 and any of the discrete barrier member 190 and the first and second cuff substrates 204, 206 may be by, for example, high pressure welding, hot air welding, heat crimping, or ultrasonic welding. Exemplary bonding methods and apparatuses may include those described in U.S. Pat. Nos. 4,854,984; 4,919,738; 5,711,847; 5,817,199; 6,123,792; 7,449,084; 6,248,195; 6,546,987; and U.S. patent application Ser. No. 14/038,812; 61/836,690; and 61/836,745.

Referring to FIG. 11B, upon exiting the third bond area 308, the topsheet substrate 286 bonded to the discrete barrier member 190, the first cuff substrate 204, and the second cuff substrate 206 may be advanced to other downstream processes.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method for manufacturing an absorbent article, wherein the absorbent article comprises a topsheet, a backsheet, a core, a discrete barrier member, a first cuff, and a second cuff, the method comprising the steps of:
advancing a first cuff substrate and a second cuff substrate in a machine direction, wherein the first cuff substrate comprises a first inner cuff edge and a first outer cuff edge and the second cuff substrate comprises a second inner cuff edge and a second outer cuff edge, and wherein the first inner cuff edge is separated from the second inner cuff edge in a cross direction by a first distance;
advancing a continuous barrier substrate in the machine direction to an infeed device;
advancing the infeed device and the continuous barrier substrate at a first velocity;
rotating a folding roll at a second velocity about an axis of rotation, wherein the second velocity is greater than the first velocity;
advancing the continuous barrier substrate onto the folding roll, wherein the folding roll comprises one or more grooves;
rotating the folding member about an axis of rotation;
cutting the continuous barrier substrate to form a discrete barrier member, wherein the barrier member comprises a leading edge portion, a trailing edge portion opposite the leading edge portion, and a central portion between the leading edge portion and the trailing edge portion;
forcing a portion of the discrete barrier member into the one or more grooves of the folding roll;
connecting the trailing edge portion of the discrete barrier member with the first cuff substrate and the second cuff substrate;
extending the trailing edge portion of the discrete barrier member in the cross direction by separating the first cuff substrate and the second cuff substrate such that the first inner cuff edge is separated from the second inner cuff edge in the cross direction by a second distance, wherein the second distance is greater than the first distance;
advancing a topsheet substrate having a first topsheet edge and a second topsheet edge opposite from the first topsheet edge in the cross direction; and
connecting the leading edge portion of the discrete barrier member with the topsheet substrate.

2. The method of claim 1, wherein each of the first cuff substrate and the second cuff substrate comprise a first surface and an opposing second surface, wherein the first surface and the second surface comprise a first edge region, an opposing second edge region, and a central region therebetween.

3. The method of claim 2, further comprising the step of folding the first cuff substrate and the second cuff substrate such that the first inner cuff edge is folded to associate with at least one of the first edge region and the central region of the first cuff substrate to form a first fold edge and the second inner cuff edge is folded to associate with at least one of the second edge region and the central region of the second cuff substrate to form a second fold edge.

4. The method of claim 1, wherein the discrete barrier member is extensible in at least one of the machine direction and the cross direction.

5. The method of claim 1, wherein the folding roll comprises a first engagement portion, a second engagement portion opposite the first engagement portion, and wherein the groove portion is between the first engagement portion and the second engagement portion.

6. The method of claim 1, wherein the step of connecting the trailing edge portion of the discrete barrier member with the first cuff substrate and the second cuff substrate includes bonding the trailing edge portion of the discrete barrier member with the first cuff substrate and the second cuff substrate.

7. The method of claim 6, wherein bonding comprises at least one of high pressure welding, hot air welding, heated crimping, and rotary ultrasonic welding

8. The method of claim 6, wherein the step of bonding the trailing edge portion of the discrete barrier member with the first cuff substrate and the second cuff substrate includes adhering the trailing edge portion of the discrete barrier member with the first cuff substrate and the second cuff substrate.

9. The method of claim 1, further comprising the step of transferring the discrete barrier member, the first cuff substrate, and the second cuff substrate to a bond roll.

10. The method of claim 1, wherein the topsheet substrate comprises one or more apertures.

11. The method of claim 1, further comprising the step of activating the topsheet substrate such that the topsheet substrate is extensible in at least one of the machine direction and the cross direction.

12. The method of claim 1, further comprising the step of folding the topsheet substrate to form a topsheet fold extending in the machine direction between the first topsheet edge and the second topsheet edge.

13. The method of claim 1, further comprising the step of positioning the topsheet substrate over at least a portion of the first cuff substrate, the second cuff substrate, and the discrete barrier member.

14. The method of claim 13, wherein the discrete barrier member is positioned between the topsheet substrate and at least one of the first cuff substrate and the second cuff substrate.

15. The method of claim 1, further comprising the step of connecting the topsheet substrate with the first cuff substrate and the second cuff substrate.

16. The method of claim 1, further comprising the step of stretching the topsheet substrate such that the first topsheet edge is separated from the second topsheet edge in the cross direction by an extended topsheet width.

17. A method for manufacturing an absorbent article, wherein the absorbent article comprises a topsheet, a backsheet, a core, a discrete barrier member, a first cuff, and a second cuff, the method comprising the steps of:
advancing a first cuff substrate and a second cuff substrate in a machine direction, wherein the first cuff substrate comprises a first inner cuff edge and an opposing first outer cuff edge, and wherein the second cuff substrate comprises a second inner cuff edge and an opposing second outer cuff edge, and wherein each of the first cuff substrate and the second cuff substrate comprise a first cuff surface and a second cuff surface such that each include an inner edge region, an opposing outer edge region, and a central region therebetween;
folding the first cuff substrate such that the first inner cuff edge is associated with at least one of the first edge region and the central region of the first cuff surface to form a first cuff fold, wherein the first cuff fold comprises a first fold edge;
folding the second cuff substrate such that the second inner cuff edge is associated with at least one of the first edge region and the central region of the first cuff surface to form a second cuff fold, wherein the second cuff fold comprises a second fold edge, wherein the first fold edge is separated by the second fold edge by a first distance in a cross direction;
advancing a continuous barrier substrate in the machine direction onto an infeed device, wherein the infeed device advances the continuous barrier substrate at a first velocity;
advancing the continuous barrier substrate onto an outer circumferential surface of a folding roll, wherein the folding roll is configured to rotate about an axis of rotation at a second velocity, and wherein the second velocity is greater than the first velocity;
cutting the continuous barrier substrate on the folding roll to form a discrete barrier member, wherein the discrete barrier member comprises a leading edge portion, a trailing edge portion opposite the leading edge portion, and a central portion between the leading edge portion and the trailing edge portion;
folding a portion of the discrete barrier substrate;
connecting the trailing edge portion of the discrete barrier member with the first cuff substrate and the second cuff substrate;
advancing a topsheet substrate having a first topsheet edge opposite from the second topsheet edge in the cross direction, wherein the first topsheet edge and the second topsheet edge extend longitudinally in the machine direction; and
connecting the leading edge portion of the discrete barrier member with the topsheet substrate.

18. A method for manufacturing an absorbent article, wherein the absorbent article comprises a topsheet, a backsheet, a core, a discrete barrier member, a first cuff, and a second cuff, the method comprising the steps of:
advancing a continuous barrier substrate in a machine direction;
positioning the continuous barrier substrate on a transfer member of an infeed device, wherein the transfer member is configured to advancing the continuous barrier substrate at a first velocity;
advancing a portion of the continuous barrier substrate onto an outer circumferential surface of a folding roll, wherein the portion of the continuous barrier substrate slips along the outer circumferential surface of the folding roll;
rotating the folding roll about an axis of rotation at a second velocity, wherein the second velocity is greater than the first velocity;
cutting the continuous barrier substrate to form a discrete barrier member;
advancing the discrete barrier substrate about the axis of rotation of the folding roll and folding a portion of the discrete barrier substrate;
advancing a cuff substrate onto a bond roll;
disposing the discrete barrier substrate on at least a portion of the cuff substrate;
connecting a trailing edge portion of the discrete barrier member with the cuff substrate;
associating a topsheet substrate with at least a portion of the discrete barrier member and the cuff substrate; and
connecting a leading edge portion of the discrete barrier member with the topsheet substrate.

19. The method of claim 18, further comprising the step of stretching the trailing edge portion of the discrete barrier member.

20. The method of claim 18, wherein the cuff substrate comprises a first cuff substrate and a second cuff substrate, and further comprising the step of folding the first cuff substrate and the second cuff substrate to form a first cuff fold and a second cuff fold.
